(19) United States
(12) Patent Application Publication
(10) Pub. No.: US 2016/0098930 A1
(43) Pub. Date: Apr. 7, 2016

(54) GRAPHICAL USER INTERFACE FOR TRANSPORTATION MANAGEMENT

(71) Applicant: Life Fight Network, LLC, Aurora, OR (US)

(72) Inventors: Justin J. Dillingham, Newberg, OR (US); Michael P. Griffiths, Aurora, OR (US); Ryan W. Swakon, West Linn, OR (US); Charles N. Hagele, Hillsboro, OR (US); Rene J. Bonnett, Meridian, ID (US); Justin L. Ahrenkiel, Sherwood, OR (US)

(21) Appl. No.: 14/506,517

(22) Filed: Oct. 3, 2014

(51) Int. Cl. G08G 5/00; G01S 19/13; G06F 3/0482; G06F 17/24; G06F 3/0484; G06F 3/0488

(52) U.S. Cl. CPC G08G 5/003; G06F 3/04847; G06F 3/04886; G06F 3/04842; G06F 3/0482; G06F 17/243; G01S 19/13

(57) ABSTRACT

A user of a mobile device receives, through the mobile device, a request to dispatch a transport vehicle to a destination. In response to the request, the mobile device provides the user with a graphical user interface, which may be used to review information regarding the request and, if required, modify the information based on the fulfillment of the request. If the graphical user interface detects that the request has been fulfilled, the graphical user interface causes the mobile device to display a form specifying information associated with fulfillment of the request and modifiable by the user. Once the user has approved the provided information or has updated this information for accuracy, the graphical user interface causes the mobile device to transmit the completed forms to an aviation management service.

### BACKGROUND

Medical transportation service providers often provide critical care transportation to seriously ill or injured patients. As a requirement to providing transportation to these seriously ill or injured patients, pilots of medical aerial transports are often required to perform various pre-flight, in-flight and post-flight procedures for each transport cycle. This may increase the time and labor required to provide such transportation services, as performance of these procedures may be difficult and may require significant data entry by the pilots prior to being able to provide medical transport. Such increases in time and labor may result in increases in response time and potentially place seriously ill or injured patients at greater risk of harm.

### BRIEF DESCRIPTION OF THE DRAWINGS

Various embodiments in accordance with the present disclosure will be described with reference to the drawings, in which:

FIG. 1 shows an illustrative example of an environment in which various embodiments can be implemented;

FIG. 2 shows an illustrative example of an environment in which various embodiments can be implemented;

FIG. 3 shows an illustrative example of a mobile device that includes a graphical user interface for determining the dispatch status for a particular aircraft and for selecting one or more options for accessing flight information in accordance with at least one embodiment;

FIG. 4 shows an illustrative example of a mobile device that includes a flight profile screen of a graphical user interface for displaying and inputting a flight profile in accordance with at least one embodiment;

FIG. 5 shows an illustrative example of a mobile device that includes a discrepancy log screen of a graphical user interface for displaying a discrepancy log associated with an aircraft in accordance with at least one embodiment;

FIG. 6 shows an illustrative example of a mobile device that includes an active leg screen of a graphical user interface for displaying and enabling input of information for a flight log in accordance with at least one embodiment;

FIG. 7 shows an illustrative example of a mobile device that includes a risk assessment checklist window of a graphical user interface for updating a risk assessment for a flight in accordance with at least one embodiment;

FIG. 8 shows an illustrative example of a mobile device that includes an en route screen of a graphical user interface for displaying information regarding a flight while en route to an assigned destination in accordance with at least one embodiment;

FIG. 9 shows an illustrative example of a mobile device that includes a graphical user interface for enabling a user to specify whether a flight was completed as assigned in accordance with at least one embodiment;

FIG. 10 shows an illustrative example of a mobile device that includes a landed status screen of a graphical user interface for enabling a user to view and provide flight information upon landing of the aircraft in accordance with at least one embodiment;

FIG. 11 shows an illustrative example of a mobile device that includes a flight form screen of a graphical user interface for reviewing one or more flight forms to be transmitted to an aviation management service in accordance with at least one embodiment;

FIG. 12 shows an illustrative example of a mobile device that includes a weight and balance screen of a graphical user interface for displaying the weight and balance of an aircraft in accordance with at least one embodiment;

FIG. 13 shows an illustrative example of a mobile device that includes a duty time screen of a graphical user interface for specifying a duty time for one or more crew members and determining whether the one or more crew members may participate in a flight in accordance with at least one embodiment;

FIG. 14 shows an illustrative example of a process for enabling a user to utilize an application to accept or decline a dispatch request in accordance with at least one embodiment;

FIG. 15 shows an illustrative example of a process for displaying and recording flight information based at least in part on user input and information provided from an aviation management service in accordance with at least one embodiment; and

FIG. 16 shows an illustrative, simplified block diagram of an example mobile device that may be used to practice at least one embodiment of the present disclosure.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Techniques described and suggested herein relate to the use of a graphical user interface for dispatch fulfillment and management of pre-flight, in-flight and post-flight procedures. In an embodiment, a pilot of a medical aerial transport vehicle (e.g., helicopter or other aircraft) receives, on a medical transport dispatch application, a dispatch request from an aviation management service or a dispatch service. The medical transport dispatch application may be installed on a computing device used by a pilot in remote (relative to the aviation management service and/or dispatch service) locations and which may be configured to enable interactions with the aviation management service and the dispatch service.

When the pilot receives, through the medical transport dispatch application, a dispatch request, the pilot may either accept or decline the request. In some embodiments, when the pilot declines a dispatch request, he/she may be required to specify, through the application, one or more reasons as to why the dispatch request was declined. In response, the medical transport dispatch application may transmit, to the dispatch service and/or aviation management service, a notification that the pilot has declined the request and the one or more reasons the pilot has denied the request. If the pilot, however, accepts the dispatch request, the medical transport dispatch application may initiate a dispatch timer and enable the pilot to perform one or more pre-flight checks. Additionally, the dispatch application may display information related to the requested dispatch, such as the flight profile, any transport vehicle discrepancies, personnel assigned to the dispatch and logistical transport information (e.g., dispatch location, destination, etc.). This information may be transmitted to the medical transport dispatch application by the aviation management service and/or the dispatch service.

Throughout the dispatch assignment, a pilot may utilize the medical transport dispatch application to view information related to the current dispatch assignment, as well as supplement or modify any information to accurately reflect the status and conditions of the transport cycle.

For instance, in some embodiments, a pilot may utilize the dispatch application to abort a current dispatch assignment at any time for any of myriad reasons (e.g., maintenance issues, weather-related issues, etc.). Additionally, a pilot may utilize the dispatch application to specify whether any additional stops were made during the dispatch assignment and record any information associated with these additional stops. Once a dispatch assignment has been completed, the dispatch application may record the time of completion and display information related to the current dispatch assignment. This enables the pilot to verify that the information recorded by the dispatch application is correct and allow the pilot to modify or supplement this information with additional data that is useful to the dispatch service and/or the aviation management service. This information may subsequently be transmitted to the aviation management service, which may utilize this information to update any flight characteristics of the medical transport vehicle (e.g., total service time, discrepancies, etc.).

In this manner, a pilot is able to perform flight procedures related to a dispatch assignment and receive critical information regarding the dispatch assignment through use of a centralized medical transport dispatch application on a mobile device. In addition, the techniques described and suggested herein facilitate additional technical advantages. For example, because the medical transport dispatch application is configured to receive pertinent aircraft and dispatch assignment information from the dispatch service and/or the aviation management service, the medical transport dispatch application assists the pilot with certain flight procedures by providing aircraft maintenance information, payload information and other information required to be input as part of these procedures. This reduces the amount of time required to perform these flight procedures and facilitates the pilot's ability to fulfill the assignment in an expeditious manner. Additionally, the medical transport dispatch application provides for greater distributed system efficiencies as manual input by the dispatch service and/or aviation management service may no longer be required since flight information and other relevant information may be transmitted to the pilot electronically and accurately. Further, any information provided by the pilot through the application is transmitted to these services as inputted by the pilot, obviating the need for a controller at the dispatch service and/or aviation management service to manually record any pilot entries.

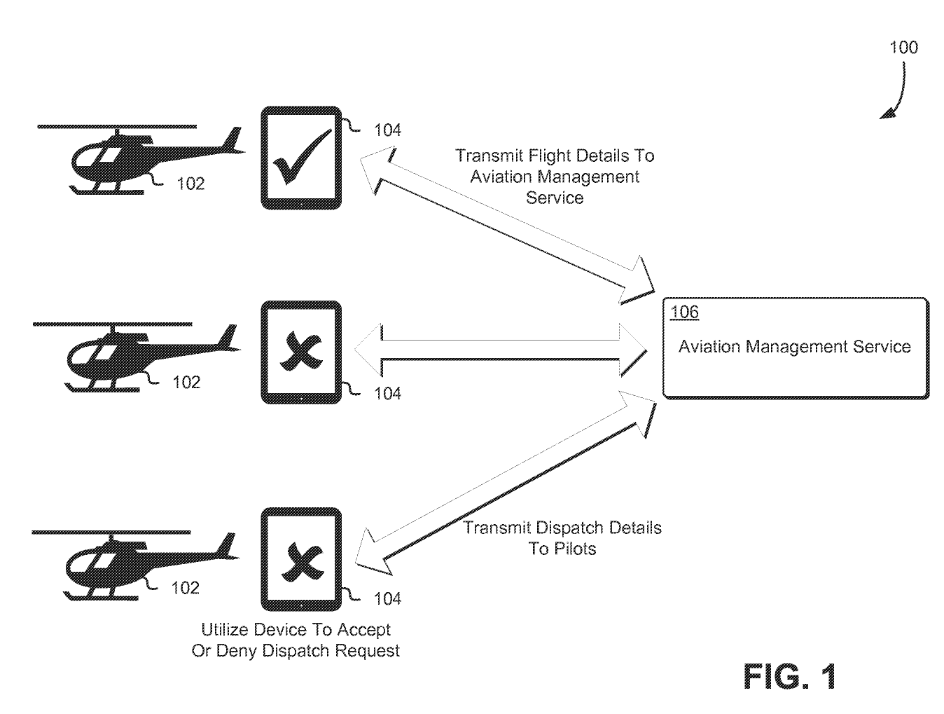

FIG. 1 shows an illustrative example of an environment 100 in which various embodiments can be implemented. In the environment 100, an aviation management service 106 or a dispatch service, transmits a dispatch request to one or more pilots through a medical transport dispatch application installed on a mobile device 104 for each of the one or more pilots. The aviation management service 106 may include one or more computing hardware resources, such as hardware servers, data storage devices, network devices, and other equipment, such as server racks, networking cables and the like. These computing hardware resources may be utilized for various purposes. For instance, the aviation management service 106 may utilize these computing hardware resources to maintain aircraft service records, track aircraft performance and flight status, perform risk assessment, schedule maintenance and other dispatch assignments and the like. The aviation management service 106 may coordinate with a dispatch service to identify any aircraft or other medical transport vehicle that may be available to fulfill any dispatch assignments.

Thus, the environment 100 includes a plurality of medical transport vehicles 102. Each medical transport vehicle 102 may be owned, leased by, or otherwise associated with the dispatch service in order to support any dispatch requests and provide rapid medical transport from a dispatch location to a medical facility (e.g., hospitals or trauma centers). The fleet of medical transport vehicles 102 may comprise one or more different aircraft types and models. For instance, the dispatch service may maintain and operate one or more rotor-wing aircraft, such as the AgustaWestland AW119 Koala, the AgustaWestland AW109 and others. Additionally, the dispatch service may maintain and operate one or more fixed-wing aircraft, such as the Pilatus PC12, King Air B200 and others. While rotor-wing and fixed-wing aircraft are used extensively throughout the present disclosure for the purpose of illustration, other transport vehicles may be coordinated utilizing the medical transport dispatch application installed on a mobile device 104. For instance, the medical transport dispatch application may be used to support a fleet of ground transportation vehicles, such as ambulances. Alternatively, the medical transport dispatch application may be utilized to coordinate other transport vehicles in a non-medical manner. For instance, the application may be utilized to coordinate, among other things, transport vehicles to/from oil rigs, charter flights, taxis, ferry services and the like.

When a new dispatch request (e.g., request to transport persons and/or materiel from one location to another) is generated by the dispatch service, the dispatch service transmits this request to the aviation management service 106, which may identify the one or more medical transport vehicles 102 in proximity to the location specified within the request. Subsequently, the aviation management service 106 may transmit the dispatch request, additional dispatch details and aircraft information to the medical transport dispatch application installed on a mobile device 104 assigned for each medical transport vehicle 102 or pilot of each medical transport vehicle 102. For instance, the aviation management service 106 may provide each user of the medical transport dispatch application with information related to the assigned flight crew, the particular assigned flight, maintenance of the assigned medical transport vehicle 102, any discrepancies associated with the assigned medical transport vehicle 102 and the like. Further, the aviation management service 106 may enable each user of the application to determine whether he/she is able to accept the dispatch request. In some embodiments, the dispatch request may be generated by the aviation management service 106, which may directly transmit the dispatch request to the application installed on the mobile device 104.

When the aviation management service 106 transmits the dispatch request and associated information to a mobile device 104, the medical transport dispatch application may produce a visual or auditory signal, which may be used to indicate that a dispatch request has been received. Accordingly, a pilot or other crew member may determine whether to accept or deny the request. If the request is denied, the pilot or other crew member may be required and/or requested to provide a rationale for denying the request. This rationale may be transmitted from the mobile device 104 to the aviation management service 106, which may record this rationale within one or more records for the particular medical transport vehicle 102 and provide this information to the dispatch service. In some embodiments, the rationale may be transmitted from the mobile device 104 directly to the dispatch service. In an embodiment, the aviation management service 106, upon receipt of the rationale from the pilot or other crew member, will transmit a new dispatch request to another pilot through his/her mobile device 104, such that this other pilot may be able to accept the request. Alternatively, or additionally, the aviation management service 106 may utilize the received rationale to determine whether the rationale is valid or not. If it is not valid, the aviation management service 106 may contact the pilot and request further information.

If a pilot or other crew member accepts the dispatch request, the pilot or other crew member may utilize one or more features of the application to perform any pre-flight procedures required to initiate the medical transport cycle. For instance, the medical transport dispatch application may include a flight profile screen, which may be accessed by selecting an iconic representation of the flight profile screen from the home screen. The flight profile screen may include information for each of the flight crew members assigned to the medical transport vehicle 102 for the dispatch assignment. This information may be obtained from the aviation management service 106. Additionally, the medical transport dispatch application may include a flight log screen, which may enable the pilot or other crew member to enter information related to the dispatch assignment, such as information related to the active flight leg.

Once the pilot has specified, through use of the medical transport dispatch application installed on the mobile device 104, that he/she has accepted the dispatch request, has entered information related to the active flight leg and is en route to the required destination, the medical transport dispatch application may display information related to the current assignment, such as information regarding the condition of the one or more patients to be retrieved from the destination, the take-off time, the current time and the like. Additionally, the application may display a landing button, which a pilot may select to specify that the medical transport vehicle 102 has arrived at the assigned destination. In response to the pilot having selected the landing button, the medical transport dispatch application may record the time of landing and the flight log for this particular leg of the flight. Additionally, the medical transport dispatch application may enable the pilot to provide information for a second flight leg from the current location of the transport 102 to the next destination (e.g., medical facility, dispatch center, etc.).

After the pilot has completed the transport cycle for this particular dispatch request, the medical transport dispatch application may cause the mobile device 104 to display a complete flight form. This flight form may detail information related to each flight leg associated with the transport cycle. This may enable the pilot to evaluate the accuracy of the information recorded by the medical transport dispatch application and make any necessary adjustments. Once the pilot has verified that the information included in the flight form is accurate, the pilot may select the end flight button on the flight form to cause the medical transport dispatch application, through the mobile device 104, to transmit the recorded flight information to the aviation management service 106 and/or the dispatch service. The aviation management service 106 and/or dispatch service may store this information in a database, wherein an entry for the particular transport vehicle 102 is updated to incorporate this information (e.g., service time, fuel usage, maintenance cycles, etc.). This database may be utilized to determine how to route future dispatch requests. For instance, if a transport vehicle, upon completion of a transport cycle, requires further maintenance, the aviation management service 106 and/or dispatch service may not route dispatch requests to such a transport vehicle until all maintenance tasks have been performed. The aviation management service 106 may generate and provide a transport cycle report, which may be provided to the dispatch service for medical transport evaluation and support. Alternatively, the dispatch service may generate and provide a transport cycle report, which may be provided to the aviation management service 106 for medical transport evaluation and support.

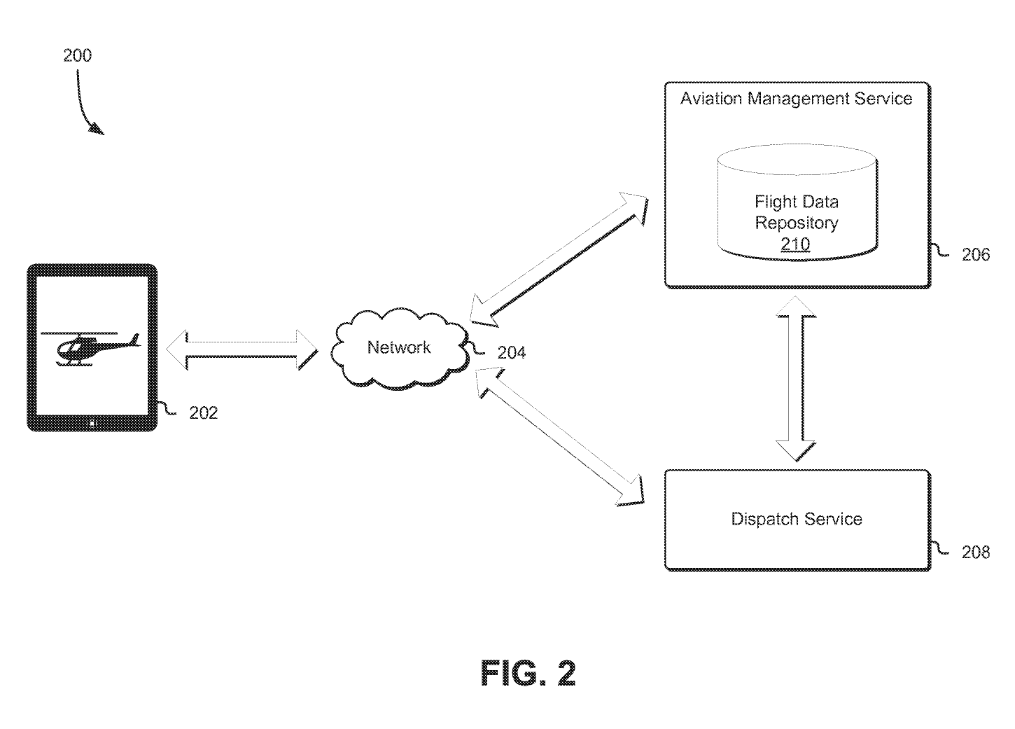

As noted above, a pilot or other crew member may utilize a medical transport dispatch application installed on a mobile device to receive dispatch requests and perform all flight procedures. Further, the information garnered from the medical transport dispatch application may be utilized, by an aviation management service and/or dispatch service, to prepare one or more reports. These reports may specify certain details of the transport vehicle utilized to perform the dispatch assignment, such as the length and duration of the transport cycle, any upcoming maintenance events and the like. Accordingly, FIG. 2 shows an illustrative example of an environment 200 in which various embodiments can be implemented. In the environment 200, a dispatch service 208 may receive notification from a medical facility, trauma center or other emergency service provider of an emergency requiring immediate medical transport of seriously ill or critically injured patients. In response to this notification, the dispatch service 208 may transmit a request to an aviation management service 206 to identify one or more medical transport vehicles that may respond to the request and transmit, through one or more communications networks 204 (e.g., WiFi, Long-Term Evolution (LTE) networks, etc.), the request to one or more mobile devices 202 associated with the identified medical transport vehicles. In some embodiments, the aviation management service 206 may receive the notification directly from the medical facility, trauma center or other emergency service provider rather than through the dispatch service 208.

Each mobile device 202 may have installed a medical transport dispatch application, which pilots and other flight crew members may utilize to record and receive information regarding a particular medical transport vehicle, discrepancies associated with the vehicle, any transport cycle and the like. When the medical transport dispatch application receives, through the mobile device 202, a dispatch request from the aviation management service 206, the medical transport dispatch application may cause the mobile device to produce a visual or auditory signal to indicate that a new dispatch request has been received. For instance, in an embodiment, the medical transport dispatch application may include, within a home screen, a status icon, which may be used to display the current status of the particular transport vehicle associated with the particular mobile device 202. When a new dispatch request is received by the medical transport dispatch application, the medical transport dispatch application may cause the status icon to blink. Additionally, the medical transport dispatch application may display an alert to the user of the mobile device 202 that a new dispatch request has been received and further generate an auditory signal which may be heard by the user of the mobile device 202. In some embodiments, the medical transport dispatch application may, through the mobile device 202, transmit the request to other devices (e.g., phones, pagers and the like) to ensure that the pilot or other crew member is notified regarding the request.

The user of the mobile device 202 may either accept or reject the dispatch request through use of the medical transport dispatch application. If the user decides to reject the dispatch request, the medical transport dispatch application may display a dialogue box, which may instruct the user to specify his/her rationale for rejecting the request. For instance, the dialogue box may include one or more buttons, each button corresponding for a reason for the rejection. These reasons may include, among other things, weather at the destination, weather en route to the destination, weather at departure, weight and balance issues, maintenance issues and the like. Once the user has provided his/her rationale for rejecting the request, the medical transport dispatch application may transmit a notification to the aviation management service 206 and/or the dispatch service 208 specifying that the request has been rejected and the appropriate rationale for the rejection.

If the user of the mobile device 202 accepts the dispatch request, the medical transport dispatch application may transmit, through the communications network 204, one or more requests to the aviation management service 206 and/or dispatch service 208, such as through one or more application programming interface (API) calls to the one or more services 206, 208, to obtain information associated with the particular transport vehicle that is to be utilized to fulfill the request. For instance, in some embodiments, the aviation management service 206 and/or dispatch service 208 may prepare a flight profile that specifies information for each flight crew member that will be involved in fulfilling the dispatch request. Additionally, the aviation management service 206 and/or dispatch service 208 may access a flight data repository 210 to identify any discrepancy logs, maintenance records and other transport vehicle characteristics that may be of use to the user of the mobile device 202. The medical transport dispatch application may also transmit one or more API calls to the aviation management service 206 and/or dispatch service 208 to request other transport information, such as a flight number and other flight details. The aviation management service 206 and the dispatch service 208 may transmit all information to the medical transport dispatch application, which may populate the display of the mobile device 202 with this information.

Once the user of the medical transport dispatch application has reviewed the supplied information and has performed all pre-flight procedures by entering, within data fields included in the medical transport dispatch application, information related to the particular transport cycle, the user may specify, through the medical transport dispatch application, that the transport vehicle has taken off and is en route to its destination. Alternatively, the medical transport dispatch application may be configured to utilize a global position system (GPS) component installed on the mobile device 202 and/or one or more accelerometers installed on the mobile device 202 to detect when the transport vehicle has departed. The medical transport dispatch application may be configured to log the time of departure and display an en route screen, which may include information regarding the dispatch request, the condition of the one or more patients to be retrieved and the like.

The user of the mobile device 202 and the medical transport dispatch application may specify, through the medical transport dispatch application, that the transport vehicle has arrived at its destination. For instance, in an embodiment, the medical transport dispatch application includes a landing button, which the user may select to specify that the transport vehicle has arrived at its destination. Alternatively, if the user forgets to utilize this button, the medical transport dispatch application may utilize the GPS component installed on the mobile device 202 and/or one or more accelerometers installed on the mobile device 202 to determine whether the transport vehicle has reached its destination. For instance, if the GPS coordinates of the destination have been provided, the medical transport dispatch application may compare the current GPS coordinates of the transport vehicle to the GPS coordinates of the destination to determine whether the transport vehicle has arrived at the destination. Alternatively, the medical transport dispatch application may utilize the GPS component of the mobile device 202 to determine if the GPS coordinates of the transport vehicle have changed over a period of time. If the GPS coordinates have not changed over a period of time, the medical transport dispatch application may determine that the transport vehicle has landed.

Once the transport vehicle has reached its destination and the medical transport dispatch application has determined (either through user input or through GPS monitoring) that the transport vehicle has indeed arrived at its destination, the medical transport dispatch application may enable the user to input additional information regarding the particular transport cycle. For instance, a user may utilize the medical transport dispatch application to specify whether any additional stops were made en route to the destination to obtain additional fuel, the number of stops made en route to the destination and to provide any receipts for expenses incurred en route to the destination. Additionally, the user may utilize the medical transport dispatch application to review a flight form, which may specify additional details regarding the transport cycle, such as the flight time, distance traveled and the like.

After the user of the mobile device 202 has verified, through the medical transport dispatch application, that the transport cycle has been completed, the medical transport dispatch application may store information associated with the transport cycle within the mobile device 202 and transmit this information to the aviation management service 206 and/or dispatch service 208, which may store this information within the flight data repository 210 for further use. For instance, the aviation management service 206 may utilize the information stored within the flight data repository 210 to generate one or more reports for each transport vehicle within a fleet associated with the dispatch service 208. These one or more reports may be provided to the aviation management service 206 and/or dispatch service 208 upon request or in response to one or more triggering events, such as an upcoming maintenance event or a discrepancy that requires immediate attention.

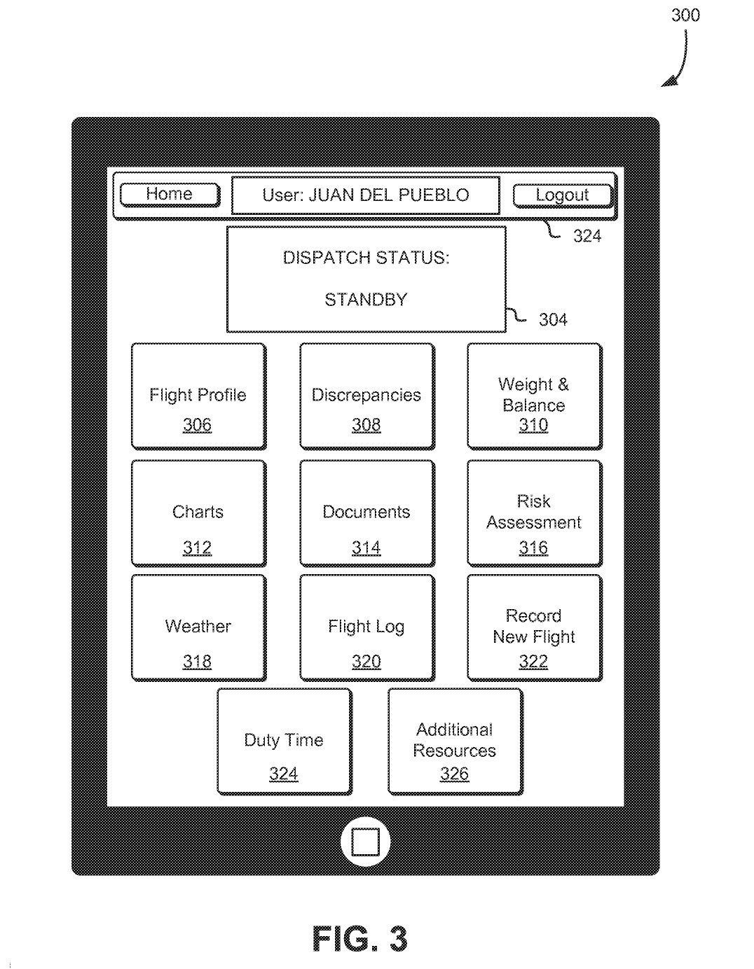

As noted above, a user of a mobile device may utilize a medical transport dispatch application to perform various transport vehicle procedures required to fulfill a dispatch request. The medical transport dispatch application may comprise a graphical user interface (GUI) which may be used by the user of the mobile device to interact with the application. Accordingly, FIG. 3 shows an illustrative example of a mobile device 300 that includes a graphical user interface for determining the dispatch status for a particular transport vehicle and for selecting one or more options for accessing flight information in accordance with at least one embodiment. The GUI for the medical transport dispatch application presented on the mobile device 302 may include various elements which a user may interact with. For instance, as noted above, the GUI may include a dispatch status window 304, which may be configured to display the current status of the transport vehicle associated with the mobile device 300. If the medical transport dispatch application receives a dispatch request from the dispatch service and/or the aviation management service, the medical transport dispatch application may utilize the dispatch status window 304 to inform the user of the mobile device 300 of the request. Further, the medical transport dispatch application may cause the mobile device 300 to produce an auditory signal (e.g., ringtone, song, etc.) which may notify the user of the incoming request.

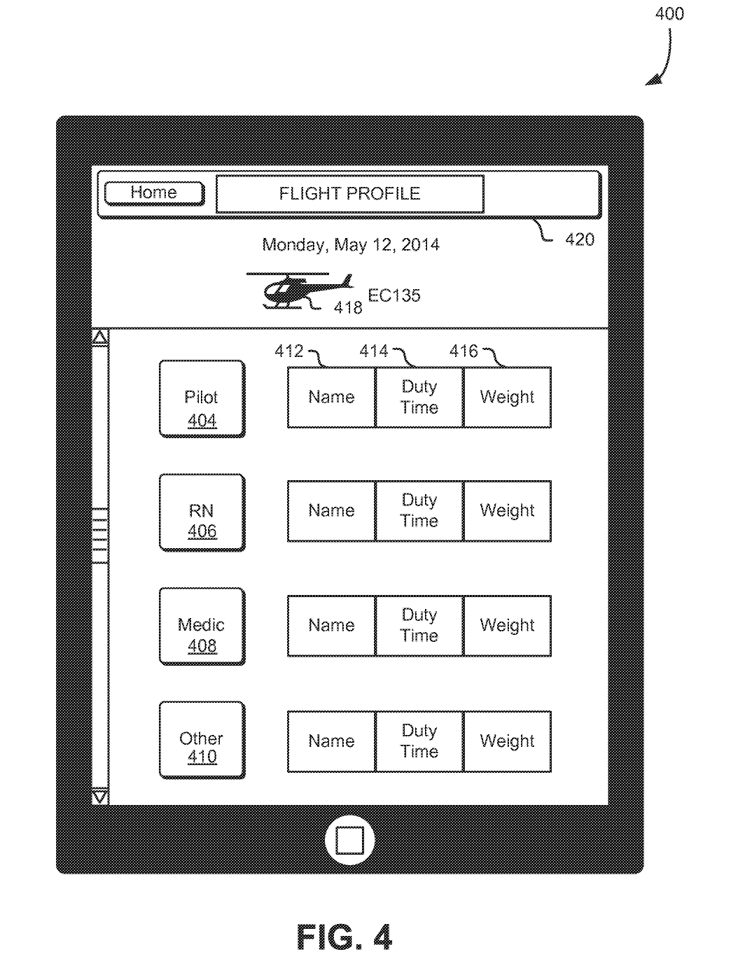

The GUI may additionally include one or more buttons, which may be used to access certain dispatch and transport vehicle information. For instance, as illustrated in FIG. 3, the medical transport dispatch application GUI includes at least nine distinct buttons: a flight profile button 306, a discrepancies button 308, a weight and balance button 310, a charts button 312, a documents button 314, a risk assessment button 316, a weather button 318, a flight log button 320 and a record new flight button 322. The flight profile button 306 may enable the user of the mobile device 300 to access the flight profile for a particular transport cycle. For instance, as will be described in greater detail in connection with FIG. 4, the flight profile screen, which may be accessed by selecting the flight profile button 306, may include information for each member of the transport vehicle crew that is to participate in the particular transport cycle.

The discrepancies button 308 may enable the user of the mobile device 300 to access a browser application, which may receive, from the aviation management service, a discrepancy log for the particular transport vehicle associated with the mobile device 300. This discrepancy log may be stored on the mobile device 300 for off-line usage by the user. Additionally, the user of the mobile device 300 may be able to submit additional discrepancies as needed to the aviation management service through use of this browser application.

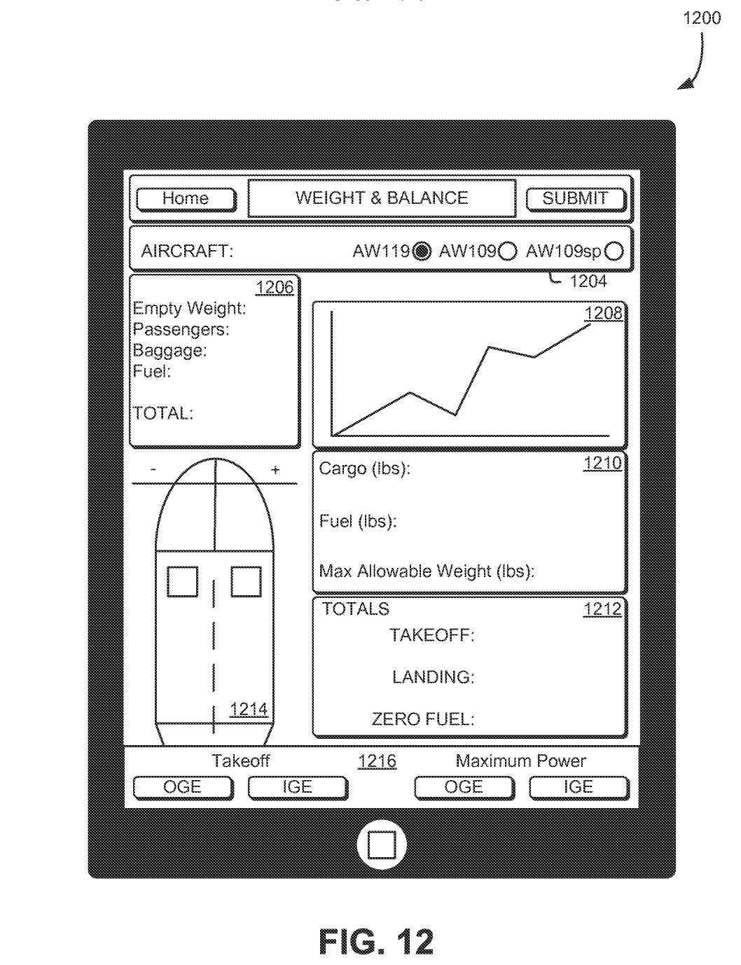

By selecting the weight and balance button 310, a user of the mobile device 300 may be able to access the weight and balance screen, as will be described in greater detail below in connection with FIG. 12. Within the weight and balance screen, a user may be able to select the appropriate transport vehicle that is to be utilized for the dispatch assignment. The medical transport dispatch application may obtain information related to the selected transport vehicle from the aviation management service and perform one or more calculations to determine the total weight of the transport vehicle throughout fulfillment of the dispatch assignment and notify the user of any balance issues. This may enable the user to redistribute any payload on the transport vehicle to ensure the transport vehicle weight distribution is balanced.

The charts button 312 and the documents button 314 may enable the user of the mobile device 300 to select and view any charts or documents associated with the transport vehicle and the particular dispatch assignment. For instance, a user may utilize the charts button 312 to access a flight chart usable to prepare a course to the assigned destination. A user may utilize the documents button 314 to access one or more documents, such as an operations manual for the transport vehicle, operations manuals for any supplemental equipment installed on the transport vehicle or included as payload and the like.

Selection of the risk assessment button 316 may cause the medical transport dispatch application installed on the mobile device 300 to display a risk assessment score for the particular dispatch assignment, as well as one or more options for updating the risk assessment score. The risk assessment score may be calculated by the aviation management service and transmitted to the medical transport dispatch application through the mobile device 300. The risk assessment score may be calculated based at least on part on certain risk factors involved with the present dispatch assignment. For instance, the dispatch service and/or aviation management service may customize the risk assessment analyze and provide, for each risk assessment item a score. This risk assessment checklist may be maintained by the aviation management service which, in turn, may evaluate the particular flight information to assign the flight a risk assessment score. A user of the mobile device 300 may access this checklist by selecting the risk assessment button 316 and may update the checklist to accurately reflect the dispatch assignment profile and obtain a new risk assessment score.

The weather button 318 may enable a user of the mobile device 300 to access current and weather reports and forecasts at the present location and at the assigned destination. For instance, if a user selects the weather button 318, the medical transport dispatch application may initiate a browser application, which may, through a communications network, access a weather forecasting service to access information regarding present and future weather conditions. This may enable the user of the mobile device 300 to determine whether the weather conditions are conducive to fulfillment of the dispatch request.

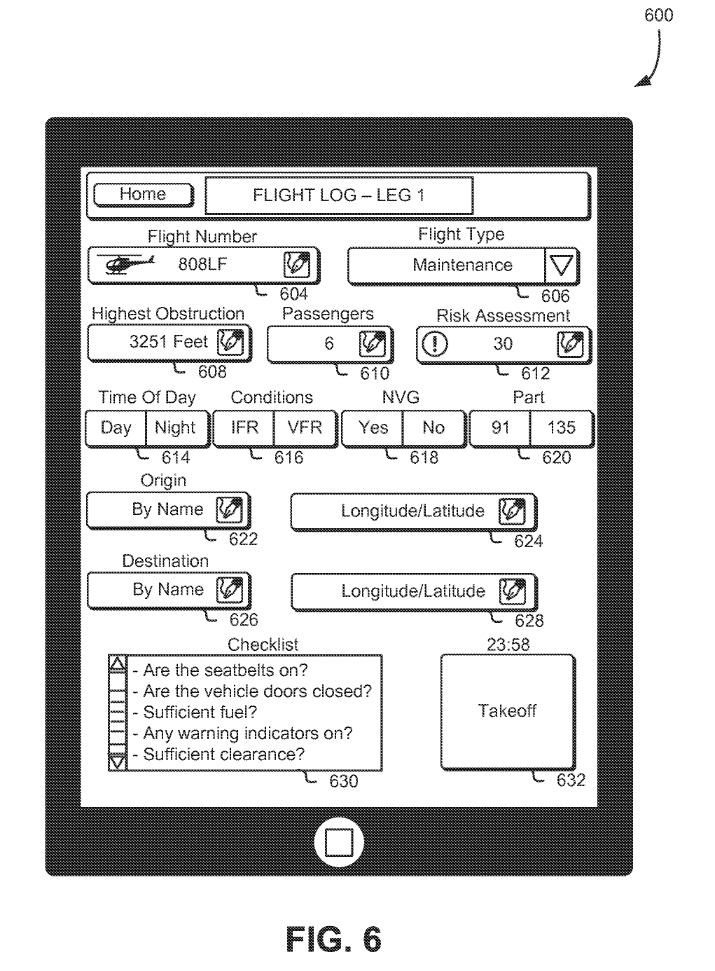

If a user selects the flight log button 320, the medical transport dispatch application may cause the mobile device 300 to display a flight log for the current dispatch assignment. For instance, as will be illustrated in connection with FIG. 6, the application may cause the mobile device 300 to display the flight log for the current leg of the transport cycle. This may enable the user to input information regarding this current leg of the transport cycle, such as the flight type, flight conditions, passengers and the like. Further, this may enable the user to provide information necessary to detail the entire transport cycle, which may subsequently be transmitted to the aviation management service. In some embodiments, information included within the flight log may be populated utilizing information received from the aviation management service in response to a dispatch request transmitted by the dispatch service to the aviation management service. If the application has not received a dispatch assignment but the user has utilized the transport vehicle for any other purpose, the user may select the record new flight button 322 to input any details related to the unassigned transport cycle.

In order to access the home screen of the medical transport dispatch application, a user may be required to provide a user name and associated password such that the aviation management service or dispatch service may determine whether the user is authorized to access the application. If the user is authorized to access the application, the user's user name may be displayed on a home bar 324 within the home screen. This home bar 324 may include one or more interface devices to enable the user to either logout of the application or return to the home screen from any other portion of the application.

As noted above, the medical transport dispatch application may include a flight profile screen, which users may utilize to identify the one or more crew members that have been assigned to the transport vehicle for the present transport cycle. Accordingly, FIG. 4 shows an illustrative example of a mobile device 400 that includes a flight profile screen of a graphical user interface for displaying and inputting a flight profile in accordance with at least one embodiment. The mobile device 400 may be the mobile device 300 described above in connection with FIG. 3. The information that may be included within the flight profile screen may be obtained from the aviation management service. For instance, when the aviation management service receives the dispatch request from the dispatch service, the request may include profiles for each crew member that is to be assigned to the transport vehicle to fulfill the request. The aviation management service may maintain, within a database, vital information for each crew member and provide this information to the application. In response to this received information, the application may be able to populate the flight profile screen.

For instance, in an embodiment, the application causes the mobile device 400 to display one or more iconic representations of the various crew members assigned to this particular dispatch assignment. For example, as illustrated in FIG. 4, the flight profile screen includes at least four different iconic representations of the crew members: a pilot icon 404, a registered nurse icon 406, a medic icon 408 and another icon 410, which may represent any other personnel that may be assigned to the particular transport vehicle for this transport cycle. For each of these crew members, the flight profile screen may specify certain vital information that may be usable to identify each crew member and enable the pilot or other crew member to incorporate this vital information for other flight procedures (e.g., weight and balance checks, etc.).

As illustrated in FIG. 4, the flight profile screen may include at least three entries for each crew member assigned to the transport vehicle for the transport cycle: a name entry 412, a duty time entry 414 and a weight entry 416, although not all embodiments may include all such entries and may, in some instances include additional and/or alternative entries. The name entry 412 may include the first name and last name of the particular crew member. The duty time entry 414 may specify the clock time at which the crew member has been activated for duty in assisting in fulfillment of the present dispatch assignment. The weight entry 416 may specify the weight of the particular crew member and may be expressed in any unit of measurement (e.g., kilograms, pounds, etc.).

The flight profile screen may further include an iconic representation of the transport vehicle 418 that is to be utilized to fulfill the dispatch assignment. This may enable the user of the mobile device 400 to quickly identify the transport vehicle to be utilized and obtain any necessary information for performance of any flight procedures prior to, during and after fulfillment of the dispatch assignment. The flight profile screen may additionally include a home bar 420, which may specify the active screen displayed on the mobile device 400 and may include a home button, which may be utilized to return to the home screen illustrated in FIG. 3.

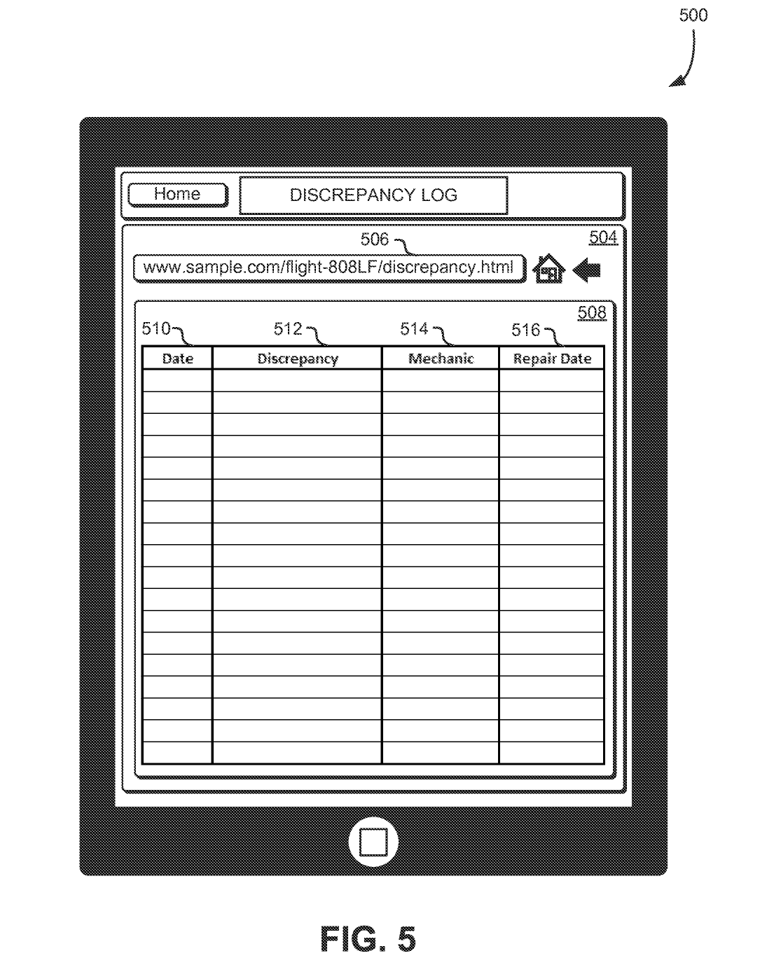

As noted above, the medical transport dispatch application may include a discrepancies log screen, which users may utilize to identify any discrepancies and/or repairs to the transport vehicle over the service life of the vehicle. Accordingly, FIG. 5 shows an illustrative example of a mobile device 500 that includes a discrepancy log screen of a graphical user interface for displaying a discrepancy log associated with a transport vehicle in accordance with at least one embodiment. The mobile device 500 may be the mobile device 300 described above in connection with FIG. 3. When a user selects the discrepancies button from the home screen, as illustrated in FIG. 3, the medical transport dispatch application may initiate a customer browser application 504 within the discrepancy log screen. Further, the medical transport dispatch application may utilize the browser application 504 to access the discrepancy log from the aviation management serve and cause a browser window 508 to display the log. For instance, the medical transport dispatch application may populate an address bar 506 of the browser application 504 with a uniform resource locator (URL) directed towards the discrepancy log for the particular transport cycle stored within one or more repositories of the aviation management service. This may cause the mobile device 500 to communicate with one or more domain name servers to identify the particular one or more aviation management service repositories associated with the Internet Protocol (IP) address corresponding to the URL and enable the mobile device 500 to access these repositories to obtain the discrepancy log.

The discrepancy log may include several entries which may be used to specify any discrepancies associated with the transport vehicle and any repairs performed to address these discrepancies. For instance, as illustrated in FIG. 5, the discrepancy log may include at least four distinct entries: a discrepancy date entry 510, a discrepancy entry 512, a mechanic entry 514 and a repair date entry 516. The discrepancy date entry 510 may specify the date in which the particular discrepancy was discovered on the transport vehicle. The discrepancy entry 512 may be used to specify the nature of the discrepancy discovered. The mechanic entry 514 may be used to specify the name and/or signature of the mechanic that has performed the one or more repairs necessary to resolve the particular discrepancy identified in the discrepancy entry 512. The mechanic entry 514 may be blank if no mechanic has performed any repairs to address the particular discrepancy. The repair date entry 516 may specify the date in which the mechanic has performed the particular repairs to address the discrepancy. This particular entry may also be left blank if no repairs have been performed.

As noted above, if a user accepts a particular dispatch request from the aviation management service and/or dispatch service, the user may utilize the medical transport dispatch application to perform all pre-flight procedures required to fulfill the dispatch request for a particular leg of the flight. For instance, as was illustrated above in connection with FIG. 3, a user may select a flight log button or a record new flight button to access a pre-flight log for providing information regarding the current transport cycle. Accordingly, FIG. 6 shows an illustrative example of a mobile device 600 that includes an active leg screen of a graphical user interface for displaying and enabling input of information for a flight log in accordance with at least one embodiment. The mobile device 600 may be the mobile device 300 described above in connection with FIG. 3. The graphical user interface may include an active leg screen, which may enable the user to utilize the mobile device 600 to input information into the transport vehicle log.

The active leg screen illustrated in FIG. 6 may include various elements that may either be populated by the user of the mobile device 600 or the aviation management service/dispatch service. The flight number entry field 604 may be utilized to input a flight number for the particular dispatch assignment. The user of the mobile device 600 may manually input the flight number within this field 604 or the field 604 may be updated by the dispatch service upon transmission of the dispatch request.

The flight type selection field 606 may be utilized to select the type of transport cycle that is currently being performed. The flight type selection field 606 may include a drop-down menu which may specify one or more different flight types (e.g., maintenance, revenue, reposition, training, etc.) for the particular transport cycle. For instance, as illustrated in FIG. 6, the user of the mobile device 600 has selected that the particular purpose of this transport cycle is for maintenance. While a drop-down menu is used extensively throughout the present disclosure for the purpose of illustration, the flight type selection field 606 may include a scroll bar to access the various options for specifying the flight type for this particular transport cycle. The highest obstruction field 608 may enable the user of the mobile device 600 to provide the altitude of the highest obstruction that will be encountered during the transport cycle. This value may be entered manually by the user. Additionally, the user may utilize the passengers field 610 to enter the number of passengers that will be transported in the transport vehicle during the particular transport cycle.

The risk assessment field 612 may include a current risk assessment score for the particular transport cycle. As noted above, the risk assessment score may be provided by the aviation management service, which may calculate the risk assessment score based at least on part on certain risk factors involved with the present dispatch assignment. If the user of the mobile device 600 opts to manually update the risk assessment score, he/she may access the risk assessment checklist by selecting the risk assessment field 612, which may cause the medical transport dispatch application to display the risk assessment checklist. This may enable the user to update the checklist to accurately reflect the dispatch assignment profile and obtain a new risk assessment score, which may be displayed on the risk assessment field 612.

The active leg screen may further include one or more fields for specifying particular transport cycle details. For instance, a user may specify the time of day through the time of day toggle 614, the transport cycle conditions (e.g., visual flight rules or instrument flight rules) through the conditions toggle 616, whether night vision goggles (NVG) are to be utilized through the NVG toggle 618 and the type of transport cycle certificate under either Federal Aviation Regulations (FAR) part 91 or 135 through the part toggle 620. The active leg screen may include additional and/or alternative fields for the transport cycle, dependent on the configuration of the application by the aviation management service and/or the dispatch service.

The user of the mobile device 600 may be able to define the origin of the transport vehicle for the particular transport cycle, as well as the destination of the transport vehicle, by either entering the origin and destination by name in an origin name field 622 and a destination name field 626, respectively or by global coordinates in an origin coordinates field 624 and a destination coordinates field 628, respectively. In an embodiment, the origin and destination information is provided by the aviation management service or the dispatch service, which may transmit this information to the mobile device 600 through one or more communications networks, such as the Internet. Thus, in some instances, the user may not be required to provide this information.

The active leg screen may further include a checklist dialogue box 630, which may include a checklist specifying the various pre-flight procedures that must be performed before the transport vehicle is permitted to depart for its destination. The user may be required to acknowledge each item specified within the checklist dialogue box 630 before he/she is permitted to utilize the takeoff button 632 to specify that the pre-flight procedures have been performed and that the transport vehicle is en route to its destination.

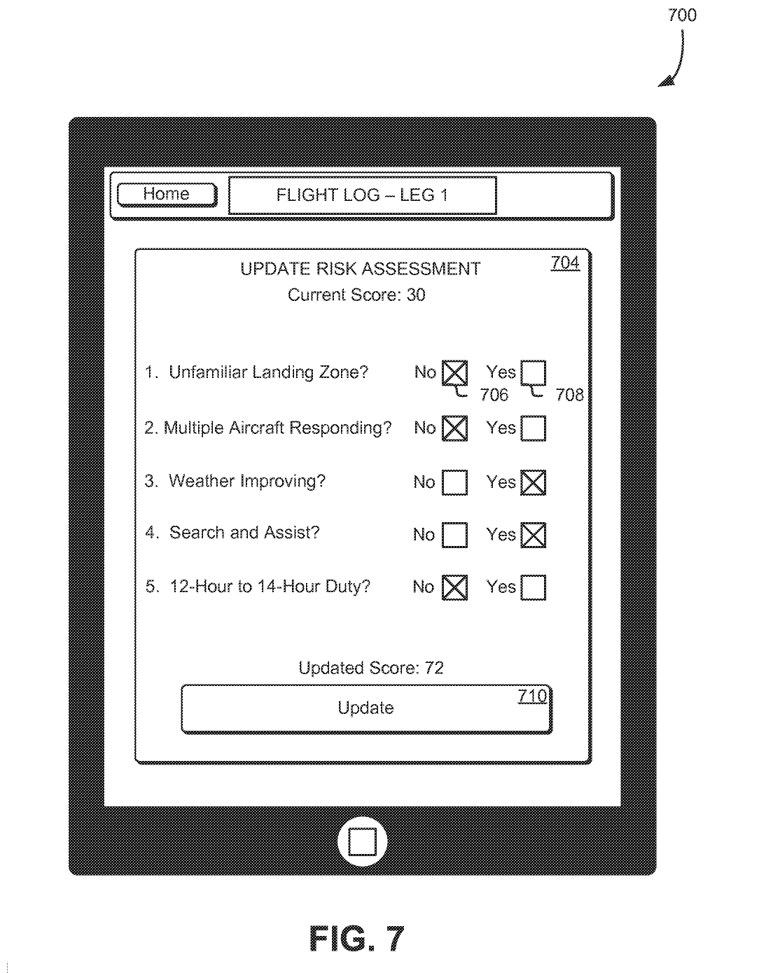

As noted above, if the user of the mobile device selects the risk assessment button within the home screen, as illustrated in FIG. 3, or selects the risk assessment field within the active leg screen, the medical transport dispatch application may cause the mobile device to display a risk assessment checklist, which the user may utilize to update the risk assessment score. Accordingly, FIG. 7 shows an illustrative example of a mobile device 700 that includes a risk assessment checklist window 704 of a graphical user interface for updating a risk assessment score for a transport cycle in accordance with at least one embodiment. The mobile device 700 may be the mobile device 300 described above in connection with FIG. 3. The graphical user interface may include a risk assessment checklist window 704, which may specify one or more particular checklist items that may be selected to update the risk assessment score. For instance, for each checklist item specified within the risk assessment checklist window 704, there may be a no checkbox 706 and a yes checkbox 708. Selecting either checkbox 706, 708 may result in an update of the risk assessment score, which may be displayed on the risk assessment checklist window 704. While yes and no checkboxes are used throughout the present disclosure for the purpose of illustration, the risk assessment checklist window 704 may utilize alternative methods to enable a user to indicate whether checklist items are to be updated in order to produce an updated risk assessment score. For example, instead of yes and no checkboxes 706, 708, the risk assessment checklist window 704 may include a drop down menu for each checklist item, which may specify various options for responding to the particular checklist item.

Once the user has completed updating the risk assessment checklist and is satisfied with the updated risk assessment score, the user may select the update button 710. In response to selection of the update button 710, the medical transport vehicle application may update the risk assessment field within the active leg screen to specify this updated risk assessment score. Further, the medical transport vehicle application may terminate the risk assessment checklist window 704 and present to the user the active leg screen, as illustrated in FIG. 6. Alternatively, if the user accessed the risk assessment checklist window 704 through selection of the risk assessment button on the home screen, the application may present to the user the home screen, as illustrated in FIG. 3.

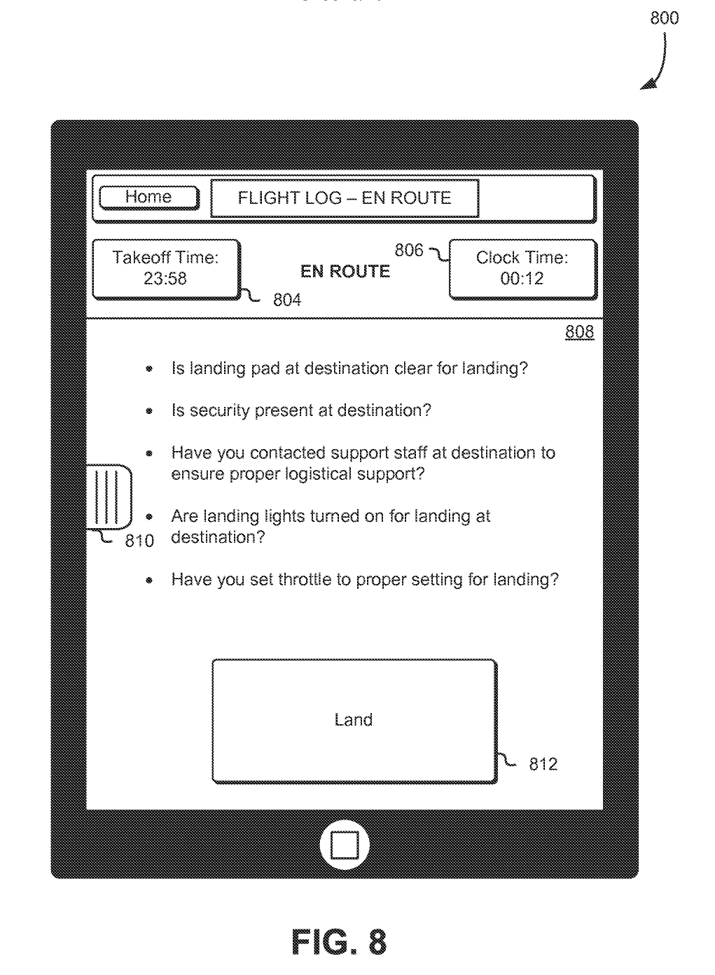

As noted above, a user of a mobile device may utilize the medical transport dispatch application to perform various pre-flight procedures and, once completed, indicate that he/she is en route to the assigned destination. For instance, as illustrated in FIG. 6, once the user of the mobile device has completed all the procedures specified within a checklist dialogue box of the active leg screen, the medical transport dispatch application may enable the user to select a takeoff button. Selection of this button may cause the medical transport dispatch vehicle to cause the mobile device to display an en route screen. Accordingly, FIG. 8 shows an illustrative example of a mobile device 800 that includes an en route screen of a graphical user interface for displaying information regarding a flight while en route to an assigned destination in accordance with at least one embodiment. The mobile device 800 may be the mobile device 300 described above in connection with FIG. 3. The en route screen may appear in response to the user having selected the takeoff button from the active leg screen. Alternatively, in an embodiment, the medical transport dispatch application utilizes a GPS component of the mobile device 800 and/or one or more accelerometers installed on the mobile device 800 to determine whether the transport vehicle has departed from its origin. If so, the application may cause the mobile device 800 to display the en route screen and log the takeoff time.

The en route screen may include various elements that the user of the mobile device 800 may utilize while en route to the assigned destination. For instance, the en route screen may include a takeoff time window 804, which may be configured to display the time at which the transport vehicle departed towards its destination. This time may be recorded in response to the user having selected the takeoff button within the active leg screen or in response to the medical transport dispatch application having detected, through the GPS component of the mobile device 800 and/or one or more accelerometers installed on the mobile device 800, that the transport vehicle has departed from its origin. The en route screen may further include a clock time window 806, which may be configured to display the current clock time. The user may utilize this displayed time to determine the amount of time that has passed since departure.

In addition to the takeoff time window 804 and the clock time window 806, the en route screen may include a text box 808, which may be used to specify certain en route information regarding the dispatch assignment, the transport vehicle and other information that may be useful to the user. This en route information may be provided to the medical transport dispatch application by either the aviation management service or the dispatch service as part of the dispatch request transmitted to the application. For instance, the en route information may include one or more checklist items that the pilot of the transport vehicle may need to perform prior to landing (e.g., engage landing lights, coordinate with support staff at destination, etc.).

The en route screen may further include a side tab 810, which the user may utilize to access information recorded previously within the active leg screen illustrated in FIG. 6. When the user of the mobile device 800 selects the side tab 810, the medical transport dispatch application may cause the information provided within the active leg screen illustrated in FIG. 6 to be displayed on the mobile device 800. Utilizing the side tab 810 again may cause the application to hide this information and again present to the user the en route screen.

The en route screen may further include a land button 812, which the user of the mobile device 800 may select to specify that the transport vehicle has landed. The medical transport dispatch application may detect that the user has selected the land button 812 and record the clock time, which is displayed on the clock time window 806 as described above. Further, this may cause the application to display a new graphical user interface, which the user may utilize to specify whether the flight has been completed as scheduled or that the flight was not completed as scheduled for one or more of myriad reasons.

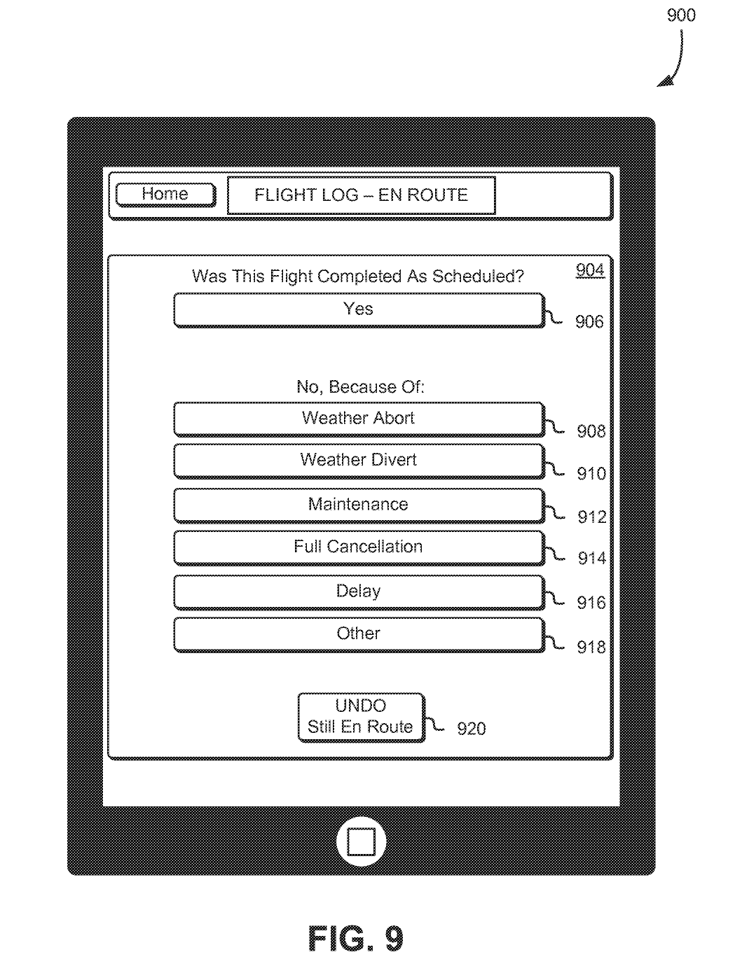

FIG. 9 shows an illustrative example of a mobile device 900 that includes a graphical user interface 904 for enabling a user to specify whether a flight was completed as assigned in accordance with at least one embodiment. The mobile device 900 may be the mobile device 300 described above in connection with FIG. 3. This graphical user interface 904 may be presented to the user of the mobile device 900 in response to the user having selected the land button described above in connection with FIG. 8. Alternatively, in an embodiment, if the medical transport dispatch application detects, through use of the GPS component installed on the mobile device 900 and/or one or more accelerometers installed on the mobile device 900, that the transport vehicle has been stationary for a certain period of time, the application may cause the mobile device 900 to display this new graphical user interface 904 and notify the user of the reasons for presenting this new interface 904 to him/her.

The graphical user interface 904 may include one or more buttons for specifying whether the dispatch assignment was completed as scheduled or not. For instance, the graphical user interface 904 may include a yes button 906, which a user of the mobile device 900 may select if the assignment was completed as scheduled. The interface 904 may further include one or more buttons, which the user of the mobile device 900 may utilize to specify that the current assignment was not completed as scheduled. For instance, as illustrated in FIG. 9, the interface 904 includes at least 6 distinct buttons, which may be utilized to provide a rationale for incompletion of the assignment. The weather abort button 908 may be selected in the event that weather conditions prevented completion of the current assignment. The weather divert button 910 may be selected if the transport vehicle had to be diverted to an alternate destination due to inclement weather conditions. The maintenance button 912 may be selected if a maintenance issue has prevented completion of the current assignment. The full cancellation button 914 may be selected if the dispatch service or aviation management service has notified the user of the mobile device 900 that the current assignment has been cancelled. The delay button 916 may be selected if there has been any delay to the current assignment for any reason, including those described above. The other button 918, when selected, may cause the mobile device 900 to display a text box, which the user may utilize to manually input one or more reasons for the assignment not being completed as scheduled.

The interface 904 may further include an undo button 920, which the user of the mobile device 900 may select to specify that the transport vehicle has not landed. This may enable the user of the mobile device 900 to return to the en route screen described above if he/she has selected the land button by mistake or to override the application if the application has determined, through use of the GPS component of the mobile device 900 and/or one or more accelerometers installed on the mobile device 900, that the transport vehicle has arrived at its destination.

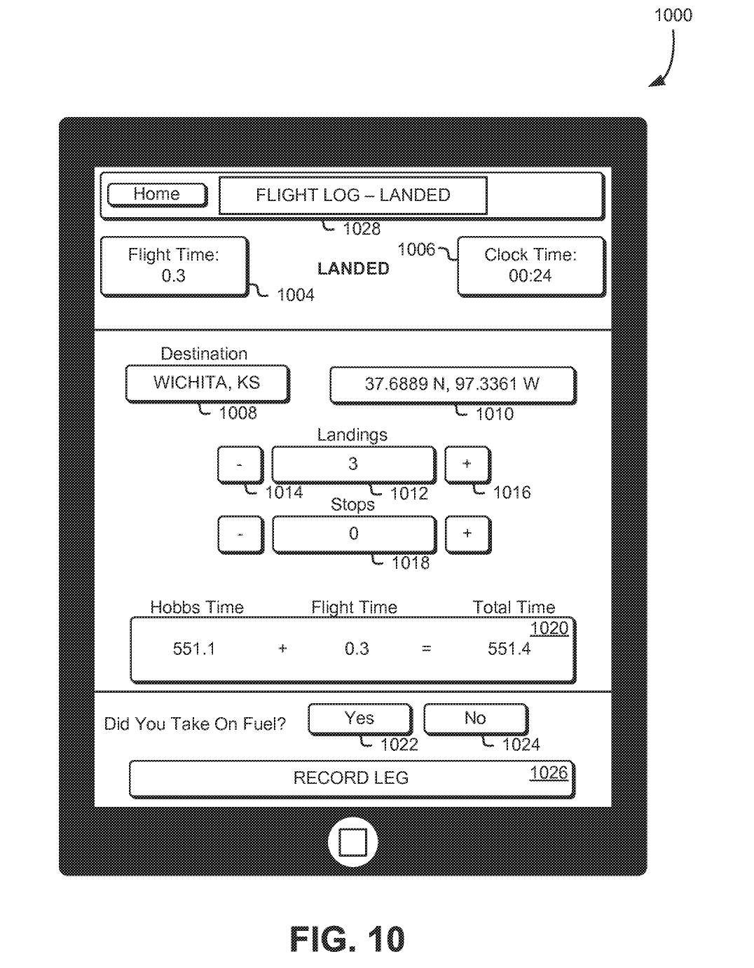

Once the user of the mobile device has specified that the current dispatch assignment has been completed as scheduled or has provided rationale for why the assignment was not completed as scheduled, the medical transport dispatch application may cause the mobile device to display a landed status screen, which may enable the user to perform any post-flight procedures and provide any additional information with regard to the transport cycle. Accordingly, FIG. 10 shows an illustrative example of a mobile device 1000 that includes a landed status screen of a graphical user interface for viewing and providing flight information upon landing of the transport vehicle in accordance with at least one embodiment. The mobile device 1000 may be the mobile device 300 described above in connection with FIG. 3. The landed status screen may appear in response to the user having selected the land button from the en route screen. Alternatively, in an embodiment, the medical transport dispatch application utilizes a GPS component of the mobile device 1000 and/or one or more accelerometers installed on the mobile device 1000 to determine whether the transport vehicle has landed. If so, the application may cause the mobile device 1000 to display the landed status screen and calculate the total transport cycle time.

The landed status screen that may be displayed on the mobile device 1000 may include various elements that may enable a user of the mobile device 1000 to perform the aforementioned post-flight procedures and provide information to the user. For instance, as illustrated in FIG. 10, the landed status screen may include a flight time window 1004, which may be configured to display the total time elapsed from the origin departure to arrival at the destination. As noted above, the medical transport dispatch application may record the takeoff time when the application detects that the transport vehicle has departed from the dispatch location. Furthermore, the application may also record the landing time at which the transport vehicle arrived at the destination. Utilizing these two values, the application may calculate the total time elapsed for the transport cycle. The landed status screen may also include a clock time window 1006, which may be configured to display the current clock time.

The destination name window 1008 and the destination coordinate window 1010 may be configured to display the current location of the transport vehicle. For instance, the destination name window 1008 may be configured to specify the city name, state and other geographical names associated with the current location of the transport vehicle. In some embodiments, the medical transport dispatch application may utilize the GPS component of the mobile device 1000 to identify the nearest city to the location of the transport vehicle and display this city name in the destination name window 1008. Similarly, the application may utilize the

GPS component of the mobile device 1000 to obtain the geographical coordinates of the location where the transport vehicle is currently located. These coordinates may be displayed in the destination coordinate window 1010.

The landed status screen may further include a landings window 1012, which may be configured to display the number of landings performed by the transport vehicle en route to the current destination. The landings window 1012 may include an add button 1014 and a subtract button 1016, which may be utilized to add landings or subtract landings, respectively, from the number of landings displayed on the landings window 1012. Similarly, the landed status screen may include a stops window 1018, which may be configured to display the number of stops performed by the transport vehicle en route to the destination. The stops window 1018 may also include its own add and subtract buttons to allow the user to modify the number of stops displayed on the stops window 1018.

As noted above, the landed status screen may include a flight time window 1004, which may be configured to display the amount of time that has elapsed during the performance of the dispatch assignment. In addition to this, the landed status screen may include a total service time window 1020, which may be configured to display the total service time of the transport vehicle.

For instance, as illustrated in FIG. 10, the total service time window 1020 may specify the Hobbs time (e.g., time the engine has been in operation) of the transport vehicle prior to the present assignment, the flight time calculated by the application and the summation of these two values. This new value may indicate the total service time of the dispatch vehicle.

The landed status screen may further enable the user of the mobile device 1000 to specify whether he/she was required to obtain fuel during the dispatch assignment. For instance, the landed status screen may include a fuel acceptance yes button 1022 and a fuel acceptance no button 1024, which may be utilized by the user to specify whether he/she has obtained fuel. If the user selects the fuel acceptance yes button 1022, the medical transport dispatch application may cause the mobile device 1000 to activate a peripheral camera. With this camera, the user may be able to record any receipts associated with the purchase of the fuel such that the user may request reimbursement from the dispatch service. Once the user has utilized the camera to record any receipts, the medical transport dispatch application may transmit these scanned receipts to the dispatch service, which may process these receipts for reimbursement. Alternatively, if the user selects the fuel acceptance no button 1024, the medical transport dispatch application may transmit, to the dispatch service, that no fuel was obtained during the transport cycle.

Once the user of the mobile device 1000 has performed all post-flight procedures specified within the landed status screen, the user may select the record leg button 1026. If the medical transport dispatch application detects that the user has selected the record leg button 1026, the application may prepare a complete flight form, which may be used to detail the present transport cycle for the user and for the dispatch service/aviation management service. Further, as will be described in greater detail below, the application may cause the mobile device 1000 to display a flight form screen, which may include one or more tables specifying the one or more details of the present transport cycle.

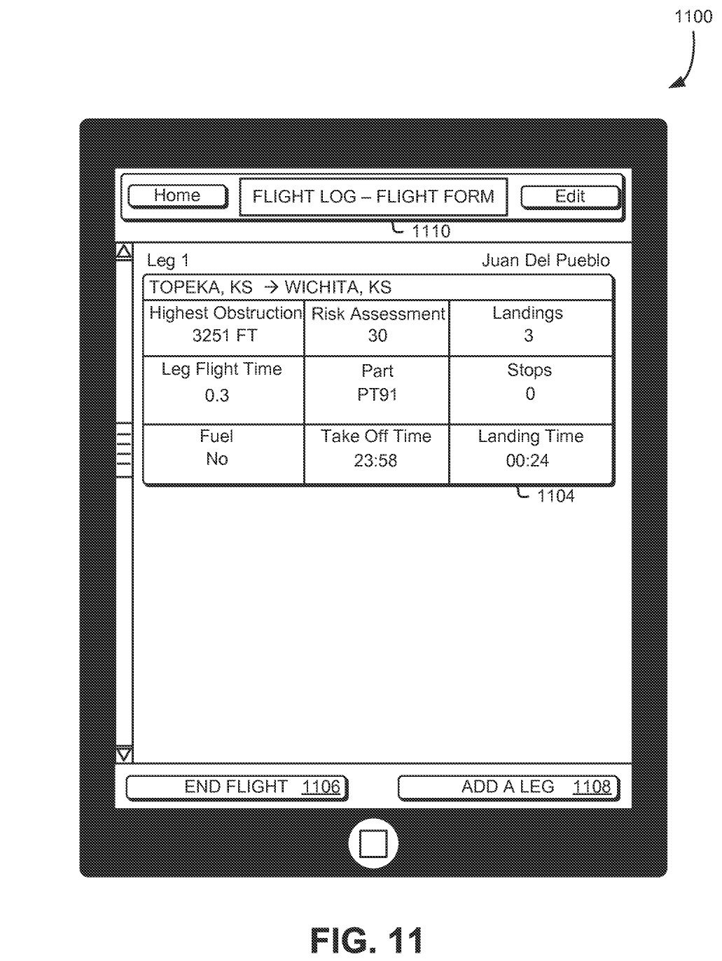

FIG. 11 shows an illustrative example of a mobile device 1100 that includes a flight form screen of a graphical user interface for reviewing one or more flight forms to be transmitted to an aviation management service in accordance with at least one embodiment. The mobile device 1100 may be the mobile device 300 described above in connection with FIG. 3. As noted above, the flight form screen may include one or more tables specifying the one or more details of the present transport cycle. Accordingly, the flight form screen, as illustrated in FIG. 11, includes a flight form table 1104. This flight form table 1104 may specify various details recorded by the application in response to user input in the prior screens, as described above. For instance, the flight form table 1104 may include the origin and destination of the transport vehicle, the altitude of the highest obstruction, the risk assessment score, the number of stops and landings during the transport cycle, the total leg flight time, the FAR part number certificate for the transport vehicle, whether fuel was obtained during the transport cycle and the takeoff and landing times. In some embodiments, the flight form table 1104 may include additional or alternative information. For instance, in some embodiments, the flight form table 1104 may specify the number of passengers on the transport vehicle, any discrepancies discovered by the user and the like.

The flight form screen may include an end flight button 1106, which a user may select to indicate that all flight procedures have been completed and that the information included within the flight form table 1104 is accurate. Selection of the end flight button 1106 may cause the medical transport dispatch application to transmit the one or more flight forms to the aviation management service, which may compile these forms and prepare a detailed report regarding the transport cycle for the dispatch service. Additionally, the flight form screen may include an add leg button 1108, which the user of the mobile device 1100 may select to manually generate additional flight forms for additional legs of a transport cycle.

As noted above, and illustrated in FIG. 3, the home screen may include a weight and balance button, which a user of the mobile device may select to access the weight and balance screen. This may enable a user to select the appropriate transport vehicle that is to be utilized for the dispatch assignment. The medical transport dispatch application may obtain information related to the selected transport vehicle from the aviation management service and perform one or more calculations to determine the total weight of the transport vehicle throughout fulfillment of the dispatch assignment and notify the user of any balance issues. Accordingly, FIG. 12 shows an illustrative example of a mobile device 1200 that includes a weight and balance screen of a graphical user interface for displaying the weight and balance of an aircraft in accordance with at least one embodiment. The mobile device 1200 may be the mobile device 300 described above in connection with FIG. 3.

The weight and balance screen may include various elements that may enable the user of the mobile device 1200 to evaluate and adjust the weight and balance of the transport vehicle prior to departure. When the user first accesses the weight and balance screen through the weight and balance button on the home screen, the user may be required to select the transport vehicle that is to be utilized to perform the dispatch assignment. For instance, the weight and balance screen may include a transport vehicle selection window 1204, which may include one or more radio buttons for each transport vehicle of the fleet of transport vehicles. Based at least in part on the user's selection of a radio button (e.g., a transport vehicle from one or more specified transport vehicles), the medical transport dispatch application may determine one or more calculations that may be performed for the selected transport vehicle.

For instance, the weight and balance screen may include a total weight window 1206, which may specify the empty weight of the selected transport vehicle, the weight of all passengers, the weight of any baggage (e.g., cargo) that is to be transported and the weight of fuel that is to be expended during the transport cycle. The empty weight of the transport vehicle may be specified within the application itself or may be obtained from the aviation management service upon selection of a radio button in the transport vehicle selection window 1204. The passenger weight may be obtained from the flight profile provided to the application by the aviation management service or dispatch service, as described above in connection with FIG. 4. The fuel weight may be computed based at least in part on the estimated transport vehicle fuel consumption rates and the estimated amount of time to complete the transport assignment. The medical transport dispatch application may utilize these three computed values to obtain a total weight for the transport vehicle, which the application may specify within the total weight window 1206.

The weight and balance screen may further include a charts window 1208, which may be used to illustrate one or more charts associated with the weight and balance of the transport vehicle. For instance, the charts window 1208 may be configured to display a chart detailing the fuel consumption of the transport vehicle over time during the assigned transport cycle. In another instance, the charts window 1208 may be configured to display a chart detailing the center of gravity envelope for the selected transport vehicle. This may enable the user of the mobile device 1200 to visualize such statistical analyses and performance analyses quickly. In an embodiment, the user of the mobile device 1200 can select the charts window 1208 to select one or more charts that may be displayed on the charts window 1208.

The weight limits window 1210 may be configured to specify the various weight limits for the transport vehicle. For instance, based at least in part on the transport vehicle selected, the application may calculate and specify, within the weight limits window 1210, the forward and aft weight limits for cargo that can be transported within the transport vehicle, the takeoff and landing fuel weight limits, the maximum allowable weight of the transport vehicle at takeoff and landing, the location of the center of gravity of the transport vehicle and the like. The weight and balance screen may further include a weight totals window 1212, which may be configured to specify the total weight of the transport vehicle for various conditions. For instance, based at least in part on the specified weight of cargo, passengers, fuel and the like, the medical transport dispatch application may calculate the total weight of the transport vehicle at takeoff, landing and with no fuel weight and specify these values within the weight totals window 1212.

The weight and balance screen may further include a weight distribution illustration 1214, which may be configured to provide a pictorial representation of the current weight distribution of the transport vehicle and the center of gravity based at least in part on the provided input from the user and the calculations performed by the medical transport dispatch application. The weight distribution illustration 1214 may utilize one or more color spectra to illustrate proper and improper weight distribution of the transport vehicle. For instance, the medical transport dispatch application may utilize red hues to demonstrate that weight distribution issues are present within the transport vehicle. Alternatively, the application may utilize green hues to demonstrate that the weight distribution is currently within tolerance. While distinct color hues are used throughout the present disclosure for the purpose of illustration, other illustration methods may be utilized to illustrate the weight distribution of the transport vehicle. For instance, in some embodiments, the medical transport dispatch application may utilize text to specify whether the weight distribution of the transport vehicle is within tolerance. For example, if the weight distribution is within tolerance, the application may utilize the word “OK.” Otherwise, the application may utilize the word “OVER” or “FAIL” to demonstrate that the current weight distribution does not satisfy the transport vehicle tolerance.

The hover charts window 1216 may include one or more buttons for accessing one or more charts related to the hover performance of the transport vehicle under various conditions. For instance, as illustrated in FIG. 12, the hover charts window 1216 may include out of ground effect (OGE) hover buttons and in ground effect (IGE) hover buttons for takeoff and maximum power conditions, although additional buttons may be provided for additional conditions. When one of the buttons is selected, the medical transport dispatch application may cause the charts window 1208 to display the selected hover chart. This may enable the user of the mobile device 1200 to view the hover chart for any given flight condition and determine whether the weight and balance of the transport vehicle are within tolerance for performing such hover maneuvers.

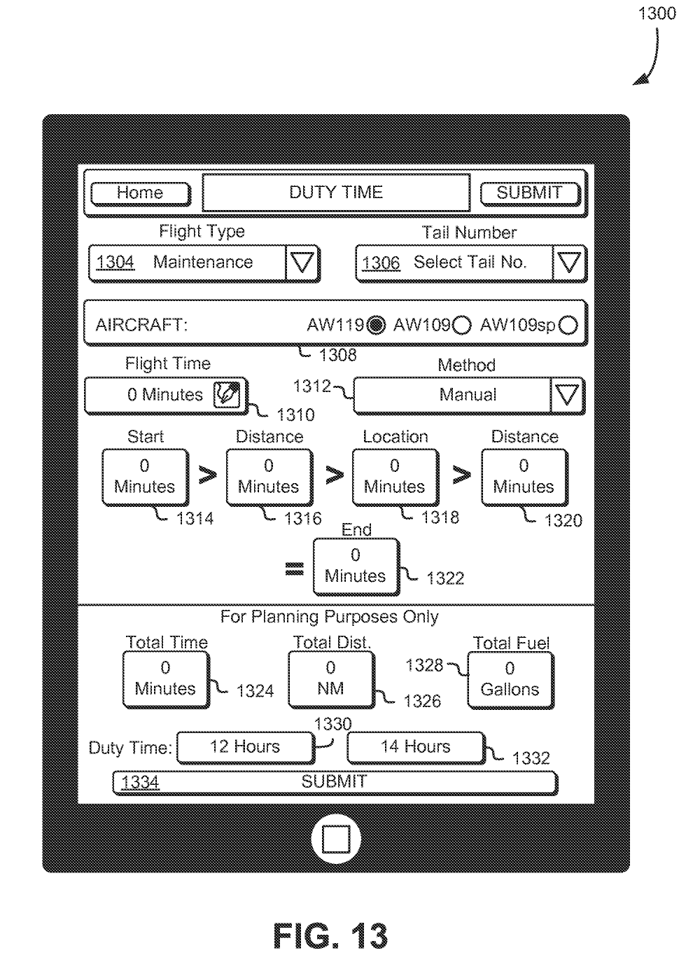

As noted above, and illustrated in FIG. 3, the home screen may include a duty time button, which a user of the mobile device may select to access the duty time screen. This may enable a user to calculate an amount of time for the particular transport cycle and determine, based at least in part on this calculation, whether the user will exceed his/her maximum duty time as established by federal law (e.g., FARs) and/or the dispatch service. The medical transport dispatch application may obtain information related to the particular transport cycle from the aviation management service and perform one or more calculations to determine the total duty time for the user of the transport vehicle for fulfillment of the dispatch assignment and notify the user of duty limitations that may inhibit his/her ability to accept the dispatch request. Accordingly, FIG. 13 shows an illustrative example of a mobile device 1300 that includes a duty time screen of a graphical user interface for specifying a duty time for one or more crew members and determining whether the one or more crew members may participate in a flight in accordance with at least one embodiment. The mobile device 1300 may be the mobile device 300 described above in connection with FIG. 3.

The duty time screen may include various elements that may enable the user of the mobile device 1300 to evaluate the amount of time required to fulfill the particular dispatch request (e.g., complete the transport cycle) and determine whether completion of this transport cycle may result in the user exceeding his/her mandatory duty time limits. When the user first accesses the duty time screen through the duty time button on the home screen, the user may be required to select the flight type, tail number and transport vehicle that is to be utilized to perform the dispatch assignment. For instance, the duty time screen may include a flight type selection field 1304, which may be utilized to select the type of transport cycle that is to be performed. The flight type selection field 1304 may include a drop-down menu which may specify one or more different flight types (e.g., maintenance, revenue, reposition, training, etc.) for the particular transport cycle. For instance, as illustrated in FIG. 13, the user of the mobile device 1300 has selected that the particular purpose of this transport cycle is for maintenance.

The duty time screen may further include a tail number selection field 1306, which may enable the user of the mobile device 1300 to select the tail number of the particular transport vehicle that may be used to complete the transport cycle. The tail number may be a unique, alphanumeric string of characters that may be used to identify a transport vehicle. This number may be assigned to the transport vehicle by a national aviation authority (e.g., FAA or any other national aviation authority). The duty time screen may additionally include a transport vehicle selection window 1308, which may include one or more radio buttons for each transport vehicle of the fleet of transport vehicles. Based at least in part on the user's selection of a radio button (e.g., a transport vehicle from one or more specified transport vehicles), the medical transport dispatch application may determine one or more calculations that may be performed to determine an estimated transport cycle time for completion. In an embodiment, when the user selects the particular transport vehicle tail number from the tail number selection field 1306, the medical transport dispatch application an associated transport vehicle, the transport vehicle associated with the selected tail number. Subsequently, the medical transport dispatch application may select the radio button within the transport vehicle selection window 1308 that corresponds to the selected tail number. In an alternative embodiment, the flight type selection field 1304, tail number selection field 1306 and the transport vehicle selection window 1308 is populated without user input based at least in part on information received from the dispatch service and/or the aviation management service through the dispatch request.

The duty time screen may further include a flight time input field 1310 and a method selection field 1312, which may be used to determine whether the flight time for the particular transport cycle is to be calculated by the medical transport dispatch application based at least in part on the received dispatch request or provided through user manual input. For instance, as illustrated in FIG. 13, the user has selected, from the method selection field 1312, that he/she will input the flight time manually within the flight time input field 1310. Accordingly, the user may select the flight time input field 1310 to manually input the estimated flight time for the transport cycle.

Based at least in part on information received from the dispatch service and/or the aviation management service with regard to the dispatch request and the selected transport vehicle, the medical transport dispatch application may populate one or more input fields to determine an estimated transport cycle time for completion of the dispatch request. For instance, the duty time screen may include a start time input field 1314, a distance to location time input field 1316, a location time input field 1318, a distance from location time input field 1320 and an end time input field 1322. The start time input field 1314 may be used to specify the amount of time that is spent on the ground prior to dispatch of the transport vehicle to the requested location. This may include the amount of time required to perform any pre-flight checks or other pre-flight procedures as required by the dispatch service, aviation management service or other authority (e.g., FAA). The distance to location time input field 1316 may be used to specify the amount of time required by the transport vehicle to travel from the dispatch location to the requested location. The time input into this field 1316 may be calculated by the medical transport dispatch application by determining an average speed for the transport vehicle selected above through the transport vehicle selection window 1308 and a flight distance from the dispatch location to the requested location (e.g., received from the dispatch service, the aviation management service and/or use of GPS mapping).

The location time input field 1318 may be automatically populated by the medical transport dispatch application. Alternatively, the user may be able to select the location time input field 1318 to manually input a location time. The distance from location time input field 1320 may be used to specify the amount of time required by the transport vehicle to travel from the requested location back to the dispatch location. The end time input field 1322 may be populated based at least in part on a sum of the times input into the prior four fields 1314, 1316, 1318, 1320. The sum may be calculated by the medical transport dispatch application. The time specified within the end time input field 1322 may be used to determine whether the user of the mobile device 1300 may accept the particular assignment.

The duty time screen may further include, for planning purposes, a total time window 1324, a total distance travelled window 1326 and a total fuel consumed window 1328. The total time window 1324 may specify the estimated total transport cycle time calculated and specified within the end time input field 1322. The total distance travelled window 1328 may specify the distance that is to be travelled during the transport cycle associated with the dispatch assignment.

As illustrated in FIG. 13, the total distance specified within the total distance travelled window 1328 may be in nautical miles, although other units of measurement may be utilized (e.g., miles, kilometers, etc.) for the total distance travelled. The total fuel consumed window 1328 may specify the estimated fuel consumption for the particular transport cycle. The information specified within these windows 1324, 1326, 1328 may be used to identify one or more procedures that may need to be performed during the transport cycle (e.g., refueling, pilot changes, etc.).

The duty time screen may include two distinct duty time windows 1330, 1332, which may specify the number of hours remaining before the user of the mobile device 1300 may no longer accept any additional dispatch requests. For instance, the dispatch service duty time window 1330 may be configured to specify the number of hours remaining, as established by the dispatch service, for the user of mobile device 1300. For example, as illustrated in FIG. 13, the dispatch service duty time window 1330 may specify that the user of the mobile device 1300 has 1200 remaining service hours before he/she can no longer accept any further dispatch requests. The initial number of service hours specified within the dispatch service duty time window 1330 may be provided by the dispatch service or the aviation management service, either of which may track the service hours used by the user of the mobile device 1300 over a particular period. The medical transport dispatch application may calculate the remaining time displayed in the dispatch service duty time window 1330 by taking the maximum allowable service hours, as defined by either the dispatch service or the aviation management service, and subtracting the service hours used by the user of the mobile device 1300 over the particular period and the calculated total time displayed in the total time window 1324 and end time input field 1322. If the resulting number displayed on the dispatch service duty time window 1330 is of a negative value, the user of the mobile device 1300 may not be permitted to accept the dispatch request.

The regulation duty time window 1332 may be configured to specify the number of hours remaining, based at least in part on a maximum number of allowable service hours defined by a regulatory agency (e.g., FAA, etc.), for the user of the mobile device 1300. The maximum number of allowable service hours defined by the regulatory agency may exceed the maximum number of allowable service hours determined by the dispatch service or aviation management service and thus, in some instances, the regulation duty time window 1332 may be used for reference. The maximum number of allowable service hours for the regulation duty time window 1332 may be programmed into the medical transport dispatch application by the dispatch service and/or aviation management service. Further, the medical transport dispatch application may calculate the remaining time displayed in the regulation duty time window 1332 using a similar set of calculations used for determining the remaining number of service hours for the dispatch service duty time window 1330, the difference being the initial number of maximum hours as defined by the regulatory agency.

Once the user of the mobile device 1300 has reviewed the information specified within the duty time screen, the user may select the submit button 1334 to provide the information to the dispatch service and/or aviation management service. The dispatch service and/or aviation management service may utilize the information provided through the duty time screen to determine whether additional dispatch requests may be transmitted to the user of the mobile device 1300 over a given time period. Further, the information may be used to analyze a user's duty time in the event that the user declines a dispatch assignment on the grounds that the dispatch assignment may cause the user to exceed his/her maximum allowable service hours for a given time period.

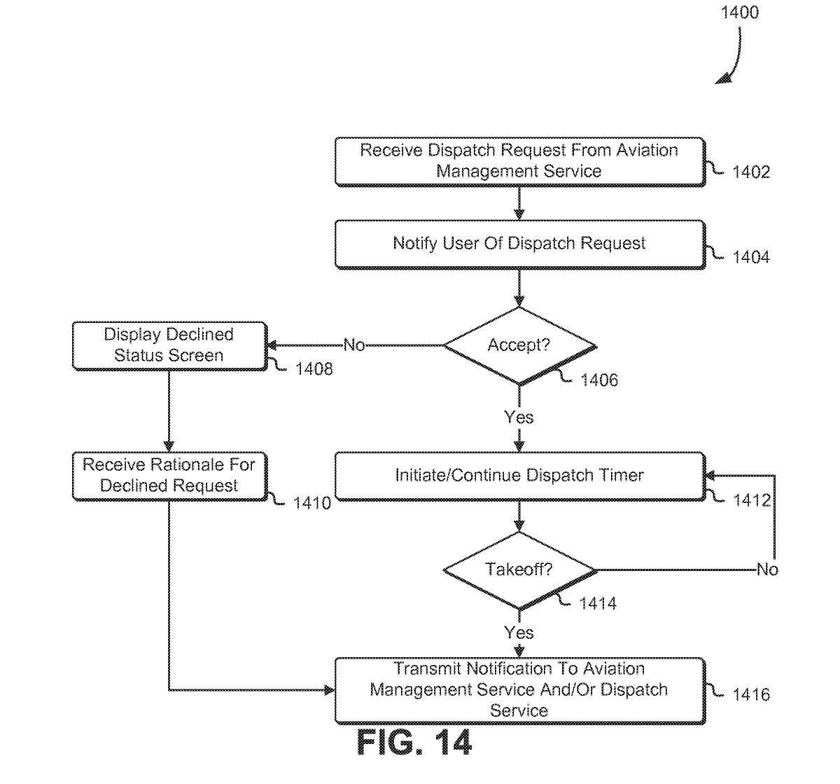

As noted above, the medical transport dispatch application may be configured to receive dispatch requests from an aviation management service and/or a dispatch service and provide any relevant information to the user of a mobile device related to the dispatch requests. Accordingly, FIG. 14 shows an illustrative example of a process 1400 for enabling a user to utilize an application to accept or decline a dispatch request in accordance with at least one embodiment. The process 1400 may be performed by a medical transport dispatch application installed on a mobile device or other computing device. The medical transport dispatch application may be configured to receive from and transmit data to the dispatch service and the aviation management service in order to provide information to the user of the mobile device and to these services as the user performs various transport cycle procedures.

When a medical emergency or other request for medical transport is received by the dispatch service, the dispatch service may coordinate this request with an aviation management service, which may be configured to obtain necessary information for each of the transport vehicles that may be in the vicinity and capable of fulfilling the request. The aviation management service, once the information necessary to fulfill the request has been compiled, may transmit this request and the necessary information to each mobile device associated with each particular crew capable of fulfilling the request. As noted above, these mobile devices may include a medical transport dispatch application, which may be installed on each of these mobile devices. Thus, the medical transport dispatch application may receive 1402, through the mobile device, the dispatch request from the aviation management service. It should be noted that in some embodiments, the request may be received from the dispatch service directly. In such embodiments, the dispatch service may provide dispatch information to the aviation management service regarding the crew that has accepted the dispatch request. This may enable the aviation management service to compile any transport cycle procedure information from this crew as it is received during and after the transport cycle.

Once the medical transport dispatch application has received this dispatch request from the aviation management service, the medical transport dispatch application may notify 1404 the user of the mobile device that a request has been received. For instance, in an embodiment, the medical transport dispatch application transmits one or more application programming interface (API) calls to a mobile device processor, which may cause the processor to transmit one or more executable instructions to various peripheral devices. These executable instructions may cause these peripheral devices to produce auditory, tactile or visual stimuli. For instance, in response to having received a dispatch request, the medical transport dispatch application may cause the mobile device to vibrate, play a sound or tone and/or cause a dispatch status window, as illustrated in FIG. 3, to blink on the mobile device screen. This may enable the user of the mobile device to determine that a dispatch request has been received and respond to the request through use of the application.

The medical transport dispatch application may determine 1406, based at least in part on the user's response to the dispatch request, whether the user has accepted the dispatch request or not. For instance, in an embodiment, a user of the mobile device utilizes the dispatch status window to specify whether he/she will accept the received dispatch request. If the user specifies that he/she has declined the dispatch request, the medical transport dispatch application may cause the mobile device to display 1408 a declined status screen. The declined status screen may be similar to the GUI illustrated in FIG. 9 in which a user may be able specify the one or more reasons as to why the request was declined. For instance, the GUI may include one or more buttons, wherein each button may correspond to a particular rationale (e.g., inclement weather at departure, inclement weather en route, weight and balance issues, etc.). Thus, when a user selects one of these buttons, the medical transport dispatch application may receive 1410 the user's rationale for declining the dispatch request and transmit 1416 notification of the declined request to the aviation management service and/or dispatch service.

If the user accepts the dispatch request, the medical transport dispatch application may initiate 1412 a dispatch timer, which may track the amount of time that has passed since the user has accepted the dispatch request and prior to the user having performed all pre-flight procedures for the current transport cycle. Additionally, the medical transport dispatch application may perform additional actions in response to the user having accepted the dispatch request. For instance, in an embodiment, the medical transport dispatch application will transmit a notification to the dispatch service and/or the aviation management service that the dispatch request has been accepted. Further, the medical transport dispatch application may cause the mobile device display to display an active leg screen, such as the active leg screen illustrated in FIG. 6. This may enable the user of the mobile device to perform all pre-flight procedures prior to takeoff.

As illustrated in FIG. 6, the active leg screen may include a takeoff button, which may be used by the user of the mobile device to specify that all pre-flight procedures have been performed and that he/she has departed for the assigned destination. The medical transport dispatch application may be configured to determine 1414 whether the transport vehicle has departed (e.g., taken off) for its assigned destination. For instance, if the user selects the takeoff button from the active leg screen, as illustrated in FIG. 6, the medical transport dispatch application may stop the dispatch timer and transmit 1416 a notification to the aviation management service and/or the dispatch service specifying that the dispatch vehicle is now en route to its destination. Further, the medical transport dispatch application may transmit information provided by the user in performing the pre-flight procedures to these services. It should be noted that in some embodiments, a user may not be required to select the takeoff button within the active leg screen to cause the application to transmit 1416 the notification to these services. For instance, in an embodiment, the medical transport dispatch application can detect, through use of one or more GPS components installed on the mobile device and/or one or more accelerometers installed on the mobile device, that the dispatch vehicle has departed from its origin.

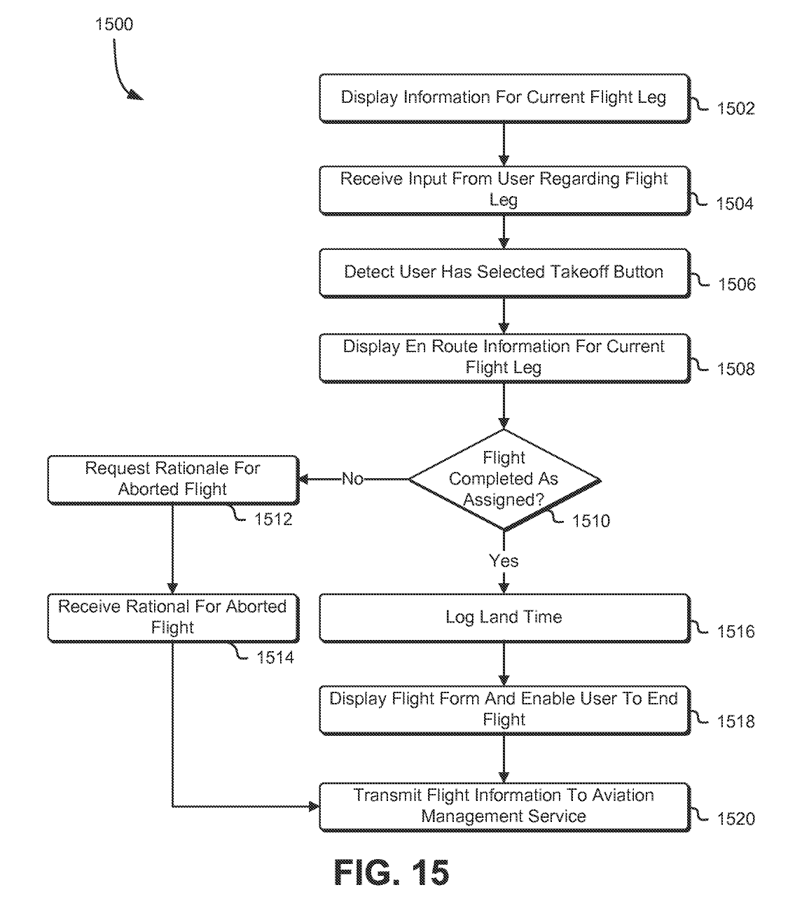

As noted above, the medical transport dispatch application may provide, to users of a mobile device, one or more GUIs, which may enable these users to perform pre-flight, en route and post-flight procedures in the course of a transport cycle. These one or more GUIs may be interconnected, such that completion of one or more elements of a GUI may cause a second GUI to be displayed on the mobile device screen. Additionally, a user may be able to revisit any previously accessed GUI in order to view previously provided information regarding the present transport cycle. Accordingly, FIG. 15 shows an illustrative example of a process 1500 for displaying and recording flight information based at least in part on user input and information provided from an aviation management service in accordance with at least one embodiment. The process 1500 may be performed by the aforementioned medical transport dispatch application, which may be installed on a mobile device or other computing device for use by one or more users associated with a transport vehicle.

When a user utilizes the medical transport dispatch application to accept a dispatch request from the aviation management service and/or the dispatch service, the application may cause the mobile device to display 1502 information for the current, active leg of the transport cycle (e.g., flight). For instance, as illustrated in FIG. 6, the medical transport dispatch application may cause the mobile device to display an active leg screen, which may include flight information, such as the flight number, origin, destination and risk assessment score. This information may be provided to the application by the aviation management service and/or the dispatch service when the dispatch request is transmitted to the mobile device.

The active leg screen may include one or more input fields which may be utilized by the user of the mobile device to provide additional flight information for the present transport assignment. For instance, as illustrated in FIG. 6, the user of the mobile device may utilize the GUI for the active leg screen to provide certain information, such as the height of the highest obstruction, the number of passengers on the transport cycle and the like. Additionally, once the user has performed all pre-flight procedures and has provided the aforementioned information, the user may be permitted to select the takeoff button to indicate that the transport vehicle has departed and is headed to the assigned destination. Thus, the medical transport dispatch application may receive 1504 input from the user for the active flight leg and further, detect 1506 that the user has selected the takeoff button.

Once the medical transport dispatch application has detected that the user has selected the takeoff button from the active leg screen, the application may cause the mobile device to display 1508 en route information for the current flight leg. For instance, as illustrated in FIG. 8, the information displayed on the mobile device may be presented in the form of an en route screen. The en route screen may specify information related to the present transport cycle, as well as other transport vehicle information, warnings and other messages from the aviation management service and/or the dispatch service. Further, the en route screen may enable the user to access any information previously provided through the active leg screen. In some embodiments, the application may cause the mobile device to display 1508 en route information without having detected that the user has selected the takeoff button from the active leg screen. For instance, as noted above, the application may utilize the one or more GPS components of the mobile device and/or one or more accelerometers installed on the mobile device to determine when the transport vehicle has moved from its current location. Such movement may indicate that the transport vehicle has departed from its origin and is now headed towards its destination.

The en route screen may include a land button, which the user of the mobile device may select to specify that the transport vehicle has landed. The medical transport dispatch application may detect that the user has selected the land button and record the clock time. Further, this may cause the application to display a new graphical user interface, such as the GUI illustrated in FIG. 9, which the user may utilize to specify whether the flight has been completed as scheduled or that the flight was not completed as scheduled for one or more of myriad reasons. Based at least in part on the user input within this new GUI, the application may determine 1510 whether the flight or transport cycle was completed as assigned. For instance, if the user has specified, through the GUI, that the transport cycle was not completed as assigned, the medical transport dispatch application may request 1512 that a rationale be provided for the aborted flight. For example, a user may be required to specify that the flight was not completed as scheduled as a result of inclement weather en route to the destination, a maintenance issue, cancellation from the dispatch service and the like. The medical transport dispatch application may receive 1514 this rationale from the user and subsequently transmit 1520 the flight information for this particular transport cycle to the aviation management service for processing and storage.

If the flight was completed as assigned, as indicated by the user through the GUI, the medical transport dispatch application may log 1516 the time at which the transport vehicle arrived at its destination and cause the mobile device to display 1518 the flight form for the completed transport cycle and enable the user to end the current flight. For instance, as illustrated in FIG. 11 and described in greater detail above, the medical transport dispatch application may cause the mobile device to display a flight form screen, which may include one or more tables specifying the one or more details of the present transport cycle. For example, the flight form table may include the origin and destination of the transport vehicle, the altitude of the highest obstruction, the risk assessment score, the number of stops and landings during the transport cycle, the total leg flight time, the FAR part number certificate for the transport vehicle, whether fuel was obtained during the transport cycle and the takeoff and landing times. In some embodiments, the flight form table may include additional or alternative information. For instance, the flight form table may specify the number of passengers on the transport vehicle, any discrepancies discovered by the user and the like.

The flight form screen may further include an end flight button, which the user may utilize to confirm that the information specified within the flight form screen is correct. If the medical transport dispatch application detects that the user has selected this button, the application may transmit 1520 the flight information to the aviation management service and/or the dispatch service. Additionally, the information may be stored within the mobile device for a certain period of time.

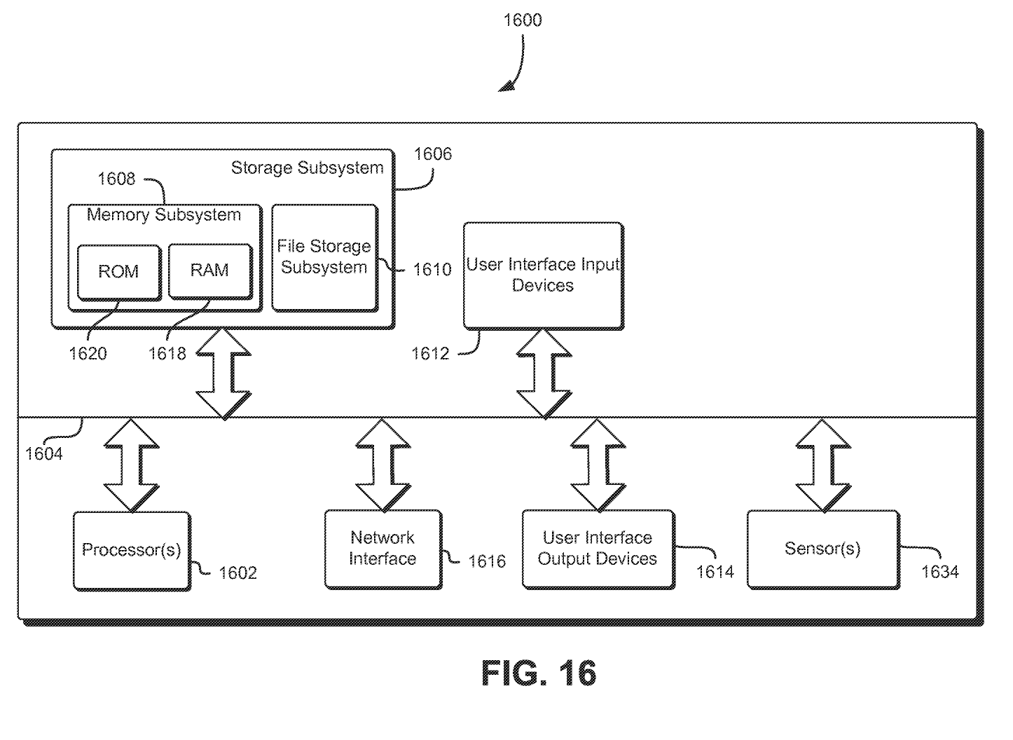

FIG. 16 is an illustrative, simplified block diagram of an example mobile device 1600 that may be used to practice at least one embodiment of the present disclosure. In various embodiments, the device system 1600 may be used to implement any of the systems illustrated herein and described above. For example, the device system 1600 may be used to implement an authentication object manager and other applications, such as a browser application, in accordance with various embodiments. As shown in FIG. 16, the device 1600 may include one or more processors 1602 that may be configured to communicate with and are operatively coupled to a number of peripheral subsystems via a bus subsystem 1604. These peripheral subsystems may include a storage subsystem 1606, comprising a memory subsystem 1608 and a file storage subsystem 1610, one or more user interface input devices 1612, one or more user interface output devices 1614, a network interface subsystem 1616, a cryptographic module 1524, comprising a memory subsystem 1530 and one or more cryptographic processors 1532. The peripheral subsystems may also include one or more sensors 1534 in addition to sensors of input devices 1612. Such sensors may include, but are not limited to, cameras, microphones, GPS sensors, accelerometers, temperature sensors and others.

The bus subsystem 1604 may provide a mechanism for enabling the various components and subsystems of device system 1600 to communicate with each other as intended. Although the bus subsystem 1604 is shown schematically as a single bus, alternative embodiments of the bus subsystem may utilize multiple busses.

The network interface subsystem 1616 may provide an interface to other device systems and networks. The network interface subsystem 1616 may serve as an interface for receiving data from and transmitting data to other systems from the device system 1600. For example, the network interface subsystem 1616 may enable transmission of authentication objects and other information, such as electronic requests to access a system (e.g., receive a webpage) and may enable receipt of responses to the requests, such as webpages or other information. The network interface subsystem 1616 may also facilitate the receipt and/or transmission of data on other networks, such as an organization's intranet and/or other networks described below. For instance, the network interface subsystem 1616 may receive dispatch request information from an aviation management service and/or a dispatch service as described in greater detail above. Additionally, the network interface subsystem 1616 may be utilized to transmit the completed flight forms, as generated through the application and, in some cases, modified by the user of the mobile device 1600, to the aviation management service and/or dispatch service as needed.

The user interface input devices 1612 may include one or more buttons, a keyboard, keypad, pointing devices, such as an integrated mouse, trackball, touchpad, or graphics tablet, a scanner, a barcode scanner, a fingerprint scanner, a retinal scanner, a touchscreen incorporated into a display, audio input devices, such as voice recognition systems, microphones, fingerprint readers, retinal scanners and other types of input devices. Further, in some embodiments, input devices may include devices usable to obtain information from other devices, such as long-term or short-term credentials for use in generating an authentication object, as described above. Input devices may include, for instance, magnetic or other card readers, one or more USB interfaces, near field communications (NFC) devices/interfaces and other devices/interfaces usable to obtain data (e.g., long-term or short-term credentials) from other devices. In general, use of the term “input device” is intended to include all possible types of devices and mechanisms for inputting information to the device system 1600.

User interface output devices 1614, if any, may include a display subsystem, a printer or non-visual displays, such as audio and/or tactile output devices, etc. Generally, the output devices 1614 may invoke one or more of any of the five senses of a user. The display subsystem may be a flat-panel device, such as a liquid crystal display (LCD), light emitting diode (LED) display, or a projection or other display device. In general, use of the term “output device” is intended to include all possible types of devices and mechanisms for outputting information from the device system 1600. The output device(s) 1614 may be used, for example, to present user interfaces to facilitate user interaction with applications performing processes described herein and variations therein, when such interaction may be appropriate. For instance, the output device(s) 1614 may be used to display the one or more transport cycle screens (e.g., active leg screen, en route screen, landed status screen, etc.) as described above. Additionally, these output device(s) 1614 may be utilized to notify the user of the mobile device 1600 of a received dispatch request.

The storage subsystem 1606 may provide a computer-readable storage medium for storing the basic programming and data constructs that may provide the functionality of at least one embodiment of the present disclosure. The applications (programs, code modules (i.e., programming modules), instructions) that, when executed by one or more processors, may provide the functionality of one or more embodiments of the present disclosure, may be stored in the storage subsystem 1606. These application modules or instructions may be executed by the one or more processors 1602. The storage subsystem 1606 may additionally provide a repository for storing data used in accordance with the present disclosure. For instance, the storage subsystem 1606 may be utilized to redundantly store the completed flight forms for a period of time upon transmission to the aviation management service and/or the dispatch service. The storage subsystem 1606 may comprise a memory subsystem 1608 and a file/disk storage subsystem 1610.

What is claimed is:

1. A computer-implemented method, comprising:
under the control of one or more computer systems configured with executable instructions,
receiving, from an aviation management service, a request to dispatch a transport vehicle, the request including information necessary for a user of a mobile device to fulfill the request;
causing the mobile device of the user to display information associated to the received request, the information modifiable by the user through a graphical user interface;
detecting, through user interaction with the graphical user interface, fulfillment of the request;
causing the mobile device of the user to display a form, the form specifying information associated with fulfillment of the request and modifiable by the user through the graphical user interface to update the information based at least in part on the user's fulfillment of the request; and
transmitting the form, from the mobile device, to the aviation management service.

2. The computer-implemented method of claim 1, further comprising:
detecting, through one or more global positioning components of the mobile device, takeoff of the transport vehicle;
causing the mobile device of the user to display en route information related to the request; and
enabling the user of the mobile device to utilize the graphical user interface to specify landing of the transport vehicle and cause the mobile device of the user to display the form.

3. The computer-implemented method of claim 1, wherein the graphical user interface includes an active leg screen, the active leg screen including one or more textual fields for modifying the information related to the received request.

4. The computer-implemented method of claim 1, further comprising:
initiating a timer upon receipt of the request to dispatch the transport vehicle;
stopping the timer upon detection, through user interaction with the graphical user interface, of the fulfillment of the request;
calculating a transport cycle time based at least in part on a total time between initiation of the timer and stoppage of the timer; and
specifying the transport cycle time on the form.

5. A computer system comprising:
one or more processors; and
memory having collectively stored therein instructions that, when executed by the computer system, cause the computer system to:
receive a request to dispatch a transport vehicle, the request including information necessary for a user to fulfill the request;
display information associated to the received request utilizing a graphical user interface, the graphical user interface usable by the user to modify the information;
detect, through user interaction with the graphical user interface, fulfillment of the request;
display one or more forms, the one or more forms specifying information associated with fulfillment of the request; and
transmit the one or more forms to an aviation management service.

6. The computer system of claim 5, wherein the graphical user interface includes one or more modifiable text fields, the one or more text fields usable by the user to modify the information and pre-populated based at least in part on the received information.

7. The computer system of claim 5, wherein the graphical user interface includes an active leg screen, the active leg screen specifying the information and including a takeoff button usable by the user to specify takeoff of the transport vehicle and access an en route screen.

8. The computer system of claim 6, wherein the instructions further cause the computer system to display the en route screen in response to the user having selected the takeoff button, the en route screen specifying additional information associated with the request and including a landing button usable by the user to specify landing of the transport vehicle.

9. The computer system of claim 7, wherein the instructions further cause the computer system to display a landed status screen in response to the user having selected the landing button, the landed status screen usable by the user to perform one or more post-flight procedures and including a record leg button, the record leg button usable to cause the computer system to display the one or more forms.

10. The computer system of claim 5, wherein the instructions further cause the computer system to:
initiate a timer upon receipt of the request to dispatch the transport vehicle;
stop the timer upon detection, through user interaction with the graphical user interface, of the fulfillment of the request;
calculate a transport cycle time based at least in part on a total time between initiation of the timer and stoppage of the timer; and
specify the transport cycle time on the form.

11. The computer system of claim 5, wherein the instructions further cause the computer system to:
utilize one or more global positioning system components to detect takeoff of the transport vehicle;
display, in response to detecting takeoff of the transport vehicle, en route information related to the request; and
enable the user to utilize the graphical user interface to specify landing of the transport vehicle, causing the one or more forms to be displayed.

12. The computer system of claim 11, wherein the instructions further cause the computer system to utilize the one or more global positioning system components to detect landing of the transport vehicle if landing of the transport has not been specified via user input.

13. A non-transitory computer-readable storage medium having collectively stored thereon executable instructions that, when executed by one or more processors of a computer system, cause the computer system to at least:
in response to having received a request to dispatch a transport vehicle to a designated destination, display information associated to the received request utilizing a graphical user interface, the information necessary for a user of the graphical user interface to fulfill the request and modifiable through use of the graphical user interface;
enable the user to utilize the graphical user interface to specify fulfillment of the request;
as a result of the request having been fulfilled, display one or more forms, the one or more forms specifying information associated with fulfillment of the request; and
transmit the one or more forms to an aviation management service.

14. The non-transitory computer-readable storage medium of claim 13, wherein the instructions further comprise instructions that, when executed by the one or more processors, cause the computer system to:
initiate a timer upon receipt of the request to dispatch the transport vehicle;
stop the timer upon detection, through user interaction with the graphical user interface, of the fulfillment of the request;
calculate a transport cycle time based at least in part on a total time between initiation of the timer and stoppage of the timer; and
specify the transport cycle time on the form.

15. The non-transitory computer-readable storage medium of claim 13, wherein the graphical user interface includes an active leg screen, the active leg screen specifying the information and including a takeoff button usable by the user to specify takeoff of the transport vehicle and access an en route screen.

16. The non-transitory computer-readable storage medium of claim 15, wherein the instructions further comprise instructions that, when executed by the one or more processors, cause the computer system to display an en route screen in response to the user having selected the takeoff button, the en route screen specifying additional information associated with the request and including a landing button usable by the user to specify landing of the transport vehicle.

17. The non-transitory computer-readable storage medium of claim 16, wherein the instructions further comprise instructions that, when executed by the one or more processors, cause the computer system to display a landed status screen in response to the user having selected the landing button, the landed status screen usable by the user to perform one or more post-flight procedures and including a record leg button, the record leg button usable to cause the computer system to display the one or more forms.

18. The non-transitory computer-readable storage medium of claim 13, wherein the instructions further comprise instructions that, when executed by the one or more processors, cause the computer system to:
utilize one or more global positioning system components to detect takeoff of the transport vehicle; and
display, in response to detecting takeoff of the transport vehicle, en route information related to the request and a landing button usable to enable the user to specify fulfillment of the request.

19. The non-transitory computer-readable storage medium of claim 18, wherein the instructions further comprise instructions that, when executed by the one or more processors, cause the computer system to utilize the one or more global positioning system components to detect fulfillment of the request.

20. The non-transitory computer-readable storage medium of claim 13, wherein the graphical user interface includes one or more modifiable text fields, the one or more text fields usable by the user to modify the information and pre-populated based at least in part on the received information.
